SEQUENCE LISTING (17 sequences)

SEQ ID NO: 1 - protein, 123 aa, Murine
EVQLVESGGGLVQPGGSLRLSCAASGFAFSSYDMSWVRQTPEKRLEWVAYISRGGGYTYYPDTVKGRFTISRDNAKNTLYLQMNSLRAEDTAVYYCSRHIYYGSSHYYAMDYWGQGTTVTVSS

SEQ ID NO: 2 - protein, 115 aa, Murine
QLVLTQSSSASASLGASVKLTCTLSRQHSTYTIEWYQQQPAKPPRYVMELKKDGSHSTGDGIPDRFSGSSSGADRYLTISSLQSEDEADYYCGVGDTIKEQFVYVFGTGTKVTVL

SEQ ID NO: 3 - protein, 12 aa, Murine
TLSRQHSTYTIE

SEQ ID NO: 4 - protein, 11 aa, Murine
LKKDGSHSTGD

SEQ ID NO: 5 - protein, 13 aa, Murine
GVGDTIKEQFVYV

SEQ ID NO: 6 - protein, 5 aa, Murine
SYDMS

SEQ ID NO: 7 - protein, 17 aa, Murine
YISRGGGYTYYPDTVKG

SEQ ID NO: 8 - protein, 14 aa, Murine
HIYYGSSHYYAMDY

SEQ ID NO: 9 - protein, 17 aa, Ebola virus
ATQVEQHHRRTDNDSTA

SEQ ID NO: 10 - DNA, 369 bp, Murine
gaggtgcaggtggtcgagtctggcggcggactggtgcagcctggcggctctctgagactgtcctgcgccgcctccggcttcgccttctcctcctacgacatgtcctgggtgcggcagacccctgagaagcggctggagtgggtggcctacatctccagaggcggcggatacacctactaccctgacaccgtgaagggccggttcaccatctcccgggacaacgccaagaacaccctgtacctgcagatgaactccctgcgggccgaggacaccgccatgtactactgctcccggcacatctactacggctcctcccactactacgccatggactactggggccagggcaccaccgtgaccgtgtcctcc

SEQ ID NO: 11 - DNA, 993 bp, Human
gcctccaccaagggcccttccgtgttccctctggccccttcctccaagtccacctccggcggcacagctgctctgggctgcctggtgaaggactacttccctgagcctgtgaccgtgagctggaactctggcgccctgaccagcggcgtgcacaccttccctgccgtgctgcagtcctccggcctgtactccctgtcctccgtggtgaccgtgccttcctcctccctgggcacccagacctacatctgcaacgtgaaccacaagccttccaacaccaaggtggacaagaaggtggagcctaagtcctgcgacaagacccatacatgcccaccctgtcctgcccctgagctgctgggcggacctagcgtgttcctgttccctcctaagcctaaggacaccctgatgatctcccggacccctgaggtgacctgcgtggtggtggacgtgtcccacgaggatcctgaggtgaagttcaattggtacgtggacggcgtggaggtgcacaacgctaagaccaagcctcgggaggagcagtacaactccacctaccgggtggtgtctgtgctgaccgtgctgcaccaggactggctgaacggcaaggaatacaagtgcaaggtctccaacaaggccctgcctgcccccatcgaaaagaccatctccaaggccaagggccagcctcgcgagcctcaggtgtacaccctgcctccctcccgggacgagctgaccaagaaccaggtgtccctgacctgtctggtgaagggcttctacccttccgatatcgccgtggagtgggagtccaacggccagcctgagaacaactacaagaccacccctcctgtgctggactccgacggctccttcttcctgtactccaagctgaccgtggacaagtcccggtggcagcagggcaacgtgttctcctgctccgtgatgcacgaggccctgcacaaccactacacccagaagagtctgagcctgtctcccggcaagtga

SEQ ID NO: 12 - DNA, 663 bp, Murine
cagctggtgctgacccagtcctcctccgcctccgcctctctgggcgcctccgtgaagctgacctgcaccctgtcccggcagcactccacctacaccatcgagtggtatcagcagcagcctgccaagcctcctagatacgtgatggagctgaagaaggacggctcccactccaccggcgacggcatccctgaccggttctccggctcctcctctggcgccgacagatacctgaccatctcctccctgcagtccgaggacgaggccgactactactgcggcgtgggcgacaccatcaaggagcagttcgtctacgtctttggcaccggcacaaaggtgaccgtgctgggccagcccaaggccgctccttccgtgaccctgttccctccttcctccgaggagctgcaggccaacaaggccaccctggtgtgcctgatctccgacttctaccctggcgccgtgaccgtggcctggaaggccgactcctcccctgtgaaggccggcgtggagacaaccaccccttccaagcagtccaacaacaagtacgccgcctcctcctacctgtccctgacccctgagcagtggaagtcccaccggtcctacagctgccaggtgacccacgagggctccaccgtggaaaagaccgtggcccctaccgagtgctcc

SEQ ID NO: 13 - DNA, 369 bp, Murine
gaggttcaggttgtggaatctggtggtggtcttgttcaacccgggggttctcttagactttcttgcgctgcttctggtttcgctttctcttcttacgatatgtcttgggtgaggcagactcctgaaaaaaggcttgagtgggtggcatatattagtaggggtggtggttacacttactaccctgatactgtgaagggaaggttcaccatttctagggataacgctaagaacaccctttaccttcagatgaactctcttagggctgaggataccgctatgtactactgctctaggcacatctactacggttcttctcactactacgctatggattattggggacagggtactactgttaccgtgtcatct

SEQ ID NO: 14 - DNA, 990 bp, Human
gcttctaccaaggggccctctgtttttcctttggctccttcatctaagtctacctctggtggtactgctgctcttggttgtttggttaaggattacttccctgagcctgttactgtgtcttggaatagtggtgctcttacttctggtgtgcatacttttccagctgtgcttcaatcttctggtctttactctctttcttctgtggtgactgtgccttcttcttctcttggtactcaaacctacatctgcaacgtgaaccacaagccttctaacaccaaagtggataagaaggttgagcctaagagctgcgataagactcatacttgtcctccatgtcctgctccagaacttcttggtggtccttctgttttcttgtttccacctaagcctaaggataccctgatgatttctaggactcctgaggttacatgcgttgtggttgatgtttctcatgaggatcctgaggtgaagttcaactggtatgttgatggtgttgaggtgcacaatgctaagactaagcctagagaggaacagtacaactctacttacagggttgtgtctgtgcttactgtgcttcatcaggattggcttaacggtaaagagtacaagtgcaaggttagcaacaaggctttgcctgctcctattgaaaagaccatctctaaggctaagggtcaacctagagaacctcaagtttacactcttccaccttctagggatgagctgactaagaatcaggtgtcacttacttgcctggtgaagggattttacccttctgatattgctgttgagtgggagtctaatggtcagcctgagaacaattacaagactactcctcctgtgctggattctgatggttcattcttcctgtactctaagctgaccgtggataagtcaagatggcaacagggtaatgtgttctcttgctctgttatgcatgaggctctgcataatcactacacccagaagtctttgtctctgtctcctggttag

SEQ ID NO: 15 - DNA, 663 bp, Murine
cagcttgtgcttacccagtcctcgagcgcttcagcttctcttggtgcttctgtgaagcttacctgcactctttctaggcagcattctacctacaccattgagtggtatcagcagcagcctgctaaacctcctagatacgtgatggaacttaagaaggatggttctcactctaccggtgatggtattcctgataggttctctggttcttcttctggtgctgatagataccttaccatttcttctcttcagtctgaggatgaggctgattactattgcggtgttggtgataccattaaggaacagttcgtgtacgttttcggaactggtactaaggttaccgttcttggacaacccaaggctgctccttctgtgactcttttccctccttcttctgaagagcttcaggctaacaaggctactcttgtgtgccttatttctgatttctaccctggtgctgttactgttgcttggaaggctgattcttctcctgttaaggctggtgttgagactactaccccttctaagcagtctaacaacaagtacgctgcttcttcttacctttctcttacccctgaacagtggaagtctcacaggtcttactcttgccaggttacccatgagggttctactgttgaaaagaccgtggctcctactgagtgttct

SEQ ID NO: 16 - protein, 330 aa, Human [sequence omitted]

SEQ ID NO: 17 - protein, 106 aa, Human
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2013/0149300 A1
(43) Pub. Date: Jun. 13, 2013

(54) MONOCLONAL ANTIBODIES WITH ALTERED AFFINITIES FOR HUMAN FCyRI, FCyRIIIa, AND C1q PROTEINS

(71) Applicants: Mapp Biopharmaceutical, Inc., San Diego, CA (US); Icon Genetics GmbH, Halle/Saale (DE)

(72) Inventors: Andrew Hiatt, San Diego, CA (US); Larry Zeitlin, Poway, CA (US)

(73) Assignees: ICON GENETICS GMBH, Halle/Saale (DE); MAPP BIOPHARMACEUTICAL, INC., San Diego, CA (US)

(21) Appl. No.: 13/628,439

(22) Filed: Sep. 27, 2012

Related U.S. Application Data
(60) Provisional application No. 61/626,420, filed on Sep. 27, 2011.

(30) Foreign Application Priority Data
Sep. 27, 2012 (US) PCT/US2012/057523

(51) Int. Cl. C07K 16/18; C07K 16/24; C07K 16/08; C07K 16/10

(52) U.S. Cl. CPC C07K 16/18; C07K 16/1018; C07K 16/10; C07K 16/24; C07K 16/08
USPC 424/133.1; 435/69.6; 435/419; 530/387.3; 536/23.53

(57) ABSTRACT

Disclosed herein are GNGN and G1/G2 antibodies that recognize and bind various FcRs and C1q. Also disclosed herein are glycan-optiminzed antibodies, predominantly of the GNGN or G1/G2 glycoform, with enhanced Fcγ receptor binding achieved through CHO, Nicotiana benthamiana and yeast manufacturing systems. Nucleic acids encoding these antibodies, as well as expression vectors and host cells including these nucleic acids are also disclosed herein. Methods and pharmaceutical compositions including the monoclonal antibodies are provided herein for the prevention and/or therapeutic treatment of viral infections, cancers and inflammatory diseases.

### PRIORITY CLAIM

This application claims the benefit of U.S. Provisional Application Ser. No. 61/626,420, filed Sep. 27, 2011, the substance of which is incorporated herein in its entirety.

### STATEMENT REGARDING FEDERALLY SPONSORED RESEARCH

The work leading to the invention that is the subject of the present application was funded in part by Grant Nos: AI61270 and AI72915 from the National Institute of Allergy and Infectious Diseases; Grant No: DAMD 17-02-2-0015 from the Department of Defense; and Grant No: 4.10007-08-RD-B from the Defense Threat Reduction Agency. Accordingly, the United States government has certain rights in the invention.

### FIELD OF THE INVENTION

The present invention relates to the field of antibodies and antigen binding fragments, specifically to antibodies that contain a substantially homogeneous glycan composition. More particularly, the present invention relates to a glycan-optimized monoclonal antibody, predominantly of either the GNGN or G1/G2 glycoform, that recognizes and binds Fc receptors and the C1q protein. Also provided are methods of producing the glycan-optimized monoclonal antibody in plants or other eukaryotic cells. Also provided are therapeutic methods that employ the monoclonal antibodies and antigen binding fragments. More particularly, the therapeutic methods include administering the glycan-optimized monoclonal antibody and/or antigen binding fragments for the prevention or treatment of human diseases including but not limited to infectious diseases (including Respiratory Syncytial virus, Ebola virus, Influenza virus), cancer (including breast cancer and B cell lymphoma) and inflammatory diseases (including rheumatoid arthritis and Alzheimer's).

### BACKGROUND OF THE INVENTION

Monoclonal antibodies (mAbs) are emerging as an important class of therapeutic agents for the treatment of human diseases such as infectious diseases, rheumatoid arthritis, and cancer [1, 2]. Currently used therapeutic mAbs for treatment or preventation of diseases are of the IgG type and are generally produced in mammalian cells (CHO cells or mouse NSO cell lines etc.). Upon recognizing an antigen and binding to the antigen contained on various targets, such as viruses, cytokines, cell surface proteins or tumor cells, mAbs can trigger various effector functions, including: 1) antibody-dependent cell-mediated cytotoxicity (ADCC); 2) complement-dependent cytotoxicity (CDC); and/or 3) signal transduction changes, e.g., induction of cell apoptosis.

It is known that appropriate glycan structures at the conserved glycosylation site (amino acid N297) of the IgG Fc domain is essential for the efficient interactions between mAbs and Fc receptors (FcR) and for the FcR-mediated effector functions, including ADCC and CDC. It was demonstrated that removing the N-glycan severely impairs ADCC and CDC [3]. On the other hand, different forms of glycosylation exert significantly different effects, some being beneficial, while others detrimental. For example, de-fucosylated, glycosylated Herceptin (trastuzumab) was shown to be at least 50-fold more active in the efficacy of Fc-gamma receptor IIIa (FcγRIIIa) mediated ADCC than Herceptin with alpha-1,6-linked fucose residues [4]. Similar results were reported for rituximab and other mAbs [5,6]. Unfortunately, recombinant mAbs are produced currently via genetic engineering, with the result that the antibody protein is present as a mixture of glycans (also known as glycoforms of the mAb), in which the more active glycoform (e.g., de-fucosylated) may be present only in minor amounts or as a component of five or more glycans. All currently marketed mAbs are only available as complex, heterogeneous glycoforms as a result of their genetic engineering origin.

Another factor in the overall efficacy of mAbs is the polymorphic nature of Fc gamma receptors (FcγR's). For example, lymphoma patients with homozygous amino acid position 158 valine/valine (V/V) alleles of FcγRIIIa (CD16a) [7] or with Fc gamma receptor FcγRIIa (CD32) amino acid position 131 histidine/histidine (H/H) alleles demonstrate a higher response rate to rituxmab treatment. The 158V allele of FcγRIIIa and the 131H allele of FcγRlla have a higher affinity to human IgG1 than does the phenylalanine (F) allele and arginine (R) allele, respectively, resulting in more effective ADCC [8]. After multivariate analysis, these two FcγR polymorphisms independently predicted longer progression free survival [9]. In light of this, it is therapeutically advantageous to purify or make recombinant mAbs with a particular glycoform optimized for affinity to particular FcγRs to enhance or minimize ADCC, CDC, or other effector functions as needed.

A typical immunoglobulin G (IgG) antibody is composed of two light and two heavy chains that are associated with each other to form three major domains connected through a flexible hinge region: the two identical antigen binding (Fab) regions and the constant (Fc) region. The IgG Fc region is a homodimer in which the two CH1 domains are paired through non-covalent interactions. The two hinge region heavy chains between CH1 and CH2 are paired through covalent bonding. The two CH2 domains are not paired but each has a conserved N-glycosylation site at Asn-297. After the antibody's recognition and binding to a target cell, ADCC and other effector functions are triggered through the binding of the antibody's Fc region to ligands such as FcγR's (FcγRI, FcγRII, and FcγRIIIa) on effector cells as well as the CI q component of complement. Essential effector functions of antibodies are dependent on appropriate glycosylation of the antibody's Fc region [10,11]. The IgG-Fc N-glycan exists naturally as a bi-antennary complex having considerable heterogeneity. The different IgG-Fc glycoforms have been shown to elicit significantly different effector functions. Jeffries et al. have demonstrated that the core structure (Man3G1cNAc2) of the N297-glycan, particularly the initial three residues (ManG1cNAc2), is essential to confer significant stability and effector activity of antibody IgG-Fc [12-14]. Structural studies suggested that the N-glycan might exert its effects mainly through stabilization of the Fc domain's conformation [13, 15, 16].

Several groups have reported that the presence of the beta-1,4-linked bisecting G1cNAc residue in the core N297-glycan could significantly enhance the antibody's ADCC activity [17-19]. Subsequent studies suggested that the lack of the alpha-1,6-linked fucose residue, rather than the presence of the bisecting G1cNAc, might play a greater role in enhancing the antibody's ADCC activity [20]. Moreover, others have reported, with various conclusions, that the terminal Gal residues may or may not positively influence the effector functions [21-24]. It is noted that these studies have involved heterogeneous glycans of the human IgG expressed in mammalian cell lines (e.g. CHO cell lines), and isolation of human IgG having a particular homogeneous glycan from this mixture is extremely difficult. Small amounts of impurities of a highly active species dramatically interferes with the results and data interpretation. Therefore, due to varying reports, unambiguous correlation of the effect on biological activity as a consequence of a specific IgG-Fc N-glycan structure remains undetermined.

An Fc receptor is a protein found on the surface of certain cells—including natural killer cells, macrophages, neutrophils, and mast cells—that contribute to the protective functions of the immune system. Its name is derived from its binding specificity for a part of an antibody known as the Fc (Fragment, crystallizable) region. Fc receptors bind to antibodies that are attached to infected cells or invading pathogens. Their activity stimulates phagocytic or cytotoxic cells to destroy microbes, or infected cells by antibody-mediated phagocytosis or antibody-dependent cell-mediated cytotoxicity.

After binding IgG, FcγRl (CD64) interacts with an accessorγ chain known as the common γ chain (γ chain), which possesses an ITAM motif that is necessary for triggering cellular activation [50]. CD64 is constitutively found on only macrophages and monocytes, but treatment of polymorphonuclear leukocytes with cytokines like IFNγ and G-CSF can induce CD64 expression on these cells [51,52]. When IgG molecules, specific for a certain antigen or surface component, bind to the pathogen with their Fab region (fragment antigen binding region), their Fc regions point outwards, in direct reach of phagocytes. Phagocytes bind those Fc regions with their Fc receptors [53]. Many low affinity interactions are formed between receptor and antibody that work together to tightly bind the antibody-coated microbe. The low individual affinity prevents Fc receptors from binding antibodies in the absence of antigen, and therefore reduces the chance of immune cell activation in the absence of infection. This also prevents agglutination (clotting) of phagocytes by antibody when there is no antigen. After a pathogen has been bound, interactions between the Fc region of the antibody and the Fc receptors of the phagocyte results in the initiation of phagocytosis. The pathogen becomes engulfed by the phagocyte by an active process involving the binding and releasing of the Fc region/Fc receptor complex, until the cell membrane of the phagocyte completely encloses the pathogen [54].

FcγRIIIA (CD16) is a low affinity Fc receptor. It is found on the surface of natural killer cells, neutrophil polymorphonuclear leukocytes, monocytes and macrophages [55]. The Fc receptor on NK cells recognize IgG that is bound to the surface of a pathogen-infected target cell and is called CD16 or FcγRIII [56]. Activation of FcγRIII by IgG causes the release of cytokines such as IFN-γ that signal to other immune cells, and cytotoxic mediators like perforin and granzyme that enter the target cell and promote cell death by triggering apoptosis. This process is known as antibody-dependent cell-mediated cytotoxicity (ADCC). FcγRIII on NK cells can also associate with monomeric IgG (i.e., IgG that is not antigen-bound). When this occurs, the Fc receptor inhibits the activity of the NK cell [57].

The collagen-like C1q molecule is a subcomponent of C1, the first component of complement, and provides a link between the innate immune system, namely the classical complement pathway, and the acquired immunity and some of its most prominent actors, the immunoglobulin classes G and M. Serum C1q is the key molecule for initiation of the classical complement cascade pathway. Its globular domains recognize the Cγ2 domain of IgG or the Cμ3 domain of IgM, especially if these antibodies are complexed with antigen and thus fixed [58-62]. However, C1q differentiates among IgG subclasses because it attaches, in terms of binding efficiency, most strongly to IgG3, followed by IgG1, but it hardly associates with IgG2 and does not react with IgG4 [63].

Cellular glycosylation engineering has emerged as an attractive approach to obtain human-like, homogeneous glycoproteins for structural studies and for biomedical applications [11, 19, 25-29]. For example, over-expression of the GnTIII gene (responsible for adding the bisecting G1cNAc to the N-glycan) in a recombinant CHO cell-line led to the production of mAbs with enhanced population of bisecting G1cNAc, which showed an increased ADCC activity (via the higher affinity binding of the mAb to FcγRIIIa) [18,19]. Expression of mAbs in a FucT-8 knock-out CHO cells (lack of the alpha-1,6-fucosyltransferase) led to non-fucosylated or low-fucose containing glycosylation states of mAbs that showed enhanced ADCC [30,31]. More recently, Gerngross [32] reported an engineered yeast Pichia pastoris system to express human-like mAbs de novo, which yielded typical bi-antennary complex type N-glycan lacking the alpha-1,6-fucose moiety [11]. In the Pichia system, rituxumab was expressed as substantially homogeneous GNGN or G2 glycoforms. Both of these glycoforms exhibited enhanced receptor binding to FcγRIIIa compared to fucosylated glycoforms with the GNGN glycoform demonstrating the best binding. Whereas the GNGN or G2 glycoforms may have enabled the enhanced FcγRIIIa interaction of this particular mouse-human IgG hybrid (mouse variable region, human IgG1 constant region containing variant amino acids [11]), the properties of a particular glycoform of an antibody are not necessarily predictable. For example, IgG2 and IgG4 antibodies have relatively low levels of receptor binding and effector functions compared to IgG1 or IgG3 antibodies regardless of the type or homogeneity of the glycoform [45]. Moreover, the variable region of the antibody can have a dramatic influence on receptor binding. For example, the 2G12 antibody [46] possesses a contorted juxtaposition of the two variable regions (referred to as “domain exchange”) and consequently has altered FcγRIIIa binding compared to rituxumab even when precisely the same glycoform is used for comparison [11, 46].

Recently, cellular glycoengineering has made major strides in the production of mAb glycoproteins with enhanced glycoforms containing predominantly a single desired glycan [11, 33] as well as greatly diminished levels of fucose in the glycan. This has addressed a long felt need for methods of producing homogeneous recombinant mAbs having a substantially homogenous glycan, and their potential use in treating a subject in need thereof. The present invention discloses mAbs with a substantially homogeneous glycan structure that is devoid of fucose and xylose residues. This mAb glycoform surprisingly confers enhanced binding affinity for human FcγRl and FcγRIIIa and a reduced binding affinity for human C1q protein. Consequently, the beneficial impact resulting from modulating a variety of effector functions is optimized.

Other and further objects, features, and advantages will be apparent from the following description of the embodiments of the invention, which are given for the purpose of disclosure.

### SUMMARY OF THE INVENTION

Provided herein are isolated monoclonal antibodies and antigen-binding fragments that contain a substantially homogeneous glycan composition. Also provided are antibodies and antigen binding fragments that contain a substantially homogenous glycan composition with a GNGN or G1/G2 glycoform and the absense of G0, G1F, G2F, GNGNX, GNGNF and GNGNXF. Unexpectedly, the substantially homogeneous glycan composition confers enhanced binding affinity for human FcγRl and FcγRIIIa and a reduced binding affinity for human C1q protein. The therapeutic or prophylactic application of these glycoforms is anticipated in circumstances where a combination of enhanced ADCC and enhanced phagocytosis embodied in one molecule is desired. Alternatively, when a combination of enhanced phagocytosis but reduced complement-dependent cytotoxicity (CDC) is desired. Further, in cases where enhanced ADCC and reduced CDC or enhanced ADCC, enhanced phagocytosis, and reduced CDC is desired.

Also provided herein are isolated antibodies or antigen-binding fragment that contain a substantially homogenous glycan composition for the prophylaxis and treatment of infectious diseases, inflammatory diseases and cancer. In some cases, the antibody or antigen-binding fragments that contain a substantially homogenous glycan composition immunospecifically bind to and neutralize Ebola virus or immunospecifically bind to the CD20 antigen to reduce inflammation and treat lymphoma or immunospecifically bind to the HER2 receptor to treat breast cancer.

Also provided herein are methods of producing antibodies or antigen-binding fragment that contain a substantially homogenous glycan composition using a plant or other eukaryotic expression system.

IgG molecules contain glycans in the CH2 domain of the Fc fragment (N-glycosylation) which are highly heterogeneous, because of the presence of different terminal sugars. The heterogeneity of Fc glycans varies with species and expression system. Fc glycans influence the binding of IgG to Fc receptors and C1q, and are therefore important for IgG effector functions. Specifically, terminal sugars such as sialic acids, core fucose, bisecting N-acetylglucosamine, and mannose residues affect the binding of IgG to the FcγRIIIa receptor and thereby influence ADCC activity. By contrast, terminal galactose residues affect antibody binding to C1q and thereby modulate CDC activity. Structural studies indicate that the presence or absence of specific terminal sugars may affect hydrophilic and hydrophobic interactions between sugar residues and amino acid residues in the Fc fragment, which in turn may impact antibody effector functions.

In one embodiment, the instant invention is drawn to a composition of a glycosylation-engineered antibody comprising immunoglobulin heavy and light chains containing a glycan, attached to heavy chain amino acid N297, wherein the substantially homogeneous glycan has terminal bisecting G1cNAc residues and is devoid of galactose, sialic acid, fucose and xylose, referred to herein as a GNGN antibody or GNGN mAb. The GNGN glycosylation-engineered antibody has altered biological activity as compared to a non-glycosylation-engineered mAb. In particular, the GNGN mAb has an increased affinity for FcγRIIIa and FcγRI and a reduced affinity for C1q. The instant invention is further drawn to the utility of the GNGN mAb for the treatment of human disease including viral infections, inflammatory disease and cancer. The instant invention is further drawn to the utility of the GNGN monoclonal antibody for infectious disease including viral infections with enhanced FcγRIIIa binding (and hence enhanced ADCC activity), enhanced FcγRI binding (and hence enhanced phagocytosis of virus with attached antibody), and minimized C1q binding (and hence a reduction of potentially inflammatory responses that could aid in viral spreading or metastases).

In a further embodiment, the instant invention is drawn to a composition of a glycosylation-engineered antibody comprising immunoglobulin heavy and light chains containing a glycan, attached to heavy chain amino acid N297, wherein the substantially homogeneous glycan has terminal bisecting G1cNAc residues and is devoid of sialic acid, fucose and xylose, referred to herein as a G1/G2 antibody or G1/G2 mAb. The G1/G2 glycosylation-engineered antibody has altered biological activity as compared to a non-glycosylation-engineered mAb. In particular, the G1/G2 mAb has an increased affinity for FcγRIIIa and FcγRI and an enhanced affinity for C1q. The instant invention is further drawn to the utility of the G1G2 mAb for the treatment of human disease including viral infections, inflammatory disease and cancer. The instant invention is further drawn to the utility of the G1/G2 monoclonal antibody for infectious disease including viral infections with enhanced FcγRIIIa binding (and hence enhanced ADCC activity), enhanced FcγRI binding (and hence enhanced phagocytosis of virus with attached antibody), and increased C1q binding (and hence an increase in inflammatory responses that could aid in virus destruction).

In another embodiment, the instant invention is drawn to a substantially homogeneous GNGN or G1/G2 glycosylation-engineered antibody comprising binding of said GNGN or G1/G2 antibody to an Fc receptor (FcR), wherein said binding is associated with an increased affinity for the FcR. An enhanced biological activity as compared to a non-glycosylation-engineered mAb results from the increased FcR binding. The instant invention is further drawn to a substantially GNGN or G1G2 mAb, wherein the mAb is an IgG antibody, and in certain embodiments, an IgG1 antibody.

In another embodiment, the instant invention is drawn to a GNGN glycosylation-engineered antibody comprising a substantially homogeneous glycan on said antibody resulting in an increased biological activity as compared to a non-glycosylation-engineered mAb.

In another embodiment, the instant invention is drawn to a G1/G2 glycosylation-engineered antibody comprising a substantially galactosylated glycan on said antibody resulting in an increased biological activity as compared to a non-glycosylation-engineered mAb.

In another embodiment, the instant invention is drawn to a method of modulating antibody-dependent cell mediated cytotoxicity (ADCC) comprising administering a glycosylation-engineered antibody.

In another embodiment, the instant invention is drawn to a method of modulating complement-dependent cytotoxicity (CDC) comprising administering a glycosylation-engineered antibody.

In another embodiment, the instant invention is drawn to a method of augmenting antibody-pathogen phagocytosis comprising administering a glycosylation-engineered antibody.

In another embodiment, the GNGN or G1/G2 glycosylation-engineered mAbs of the present invention are capable of modulated ADCC, which means an increase in biological activity relative to the non-glycosylation-engineered mAb.

In another embodiment, the GNGN or G1/G2 glycosylation-engineered mAbs of the present invention are capable of modulated CDC, which means a decrease in biological activity relative to the non-glycosylation-engineered mAb.

In another embodiment, the GNGN or G1G2 glycosylation-engineered mAbs of the present invention are capable of modulated phagocytosis of antibody-pathogen complexes or antibody-antigen complexes, which means an increase in biological activity relative to the non-glycosylation-engineered mAb.

The instant invention is further drawn to compositions wherein an antibody is a mAb, preferably an IgG antibody, and in certain embodiments IgG1 antibody. Non-exemplary antibodies contemplated include a therapeutic glycosylation-engineered GNGN or G1/G2 mAb wherein the starting antibody includes, but is not limited to, cetuximab, rituximab, muromonab-CD3, abciximab, daclizumab, basiliximab, palivizumab, infliximab, trastuzumab, gemtuzumab ozogamicin, alemtuzumab, ibritumomab tiuxetan, adalimumab, omalizumab, tositumomab, 1-131 tositumomab, efalizumab, bevacizumab, panitumumab, pertuzumab, natalizumab, etanercept, IGN101 (Aphton), volociximab (Biogen Idee and PDL BioPharm), Anti-CD80 mAb (Biogen Idee), Anti-CD23 mAb (Biogen Idel), CAT-3888 (Cambridge Antibody Technology), CDP791 (Imclone), eraptuzumab (Immunomedics), MDX-010 (Medarex and BMS), MDX-060 (Medarex), MDX-070 (Medarex), matuzumab (Merck), CP-675,206 (Pfizer), CAL (Roche), SGN-30 (Seattle Genetics), zanolimumab (Serono andGenmab), adecatumumab (Sereno), oregovomab (United Therapeutics), nimotuzumab (YM Bioscience), ABT-874 (Abbott Laboratories), denosumab (Amgen), AM 108 (Amgen), AMG 714 (Amgen), fontolizumab (Biogen Idee and PDL BioPharm), daclizumab (Biogent Idee and PDL BioPharm), golimumab (Centocor and Schering-Plough), CNTO 1275 (Centocor), ocrelizumab (Genetech and Roche), HuMax-CD20 (Genmab), belimumab (HGS and GSK), epratuzumab (Immunomedics), MLN1202 (Millennium Pharmaceuticals), visilizumab (PDL BioPharm), tocilizumab (Roche), ocrerlizumab (Roche), certolizumab pegol (UCB, formerly Celltech), eculizumab (Alexion Pharmaceuticals), pexelizumab (Alexion Pharmaceuticals and Procter & Gamble), abciximab (Centocor), ranibizimumab (Genetech), mepolizumab (GSK), TNX-355 (Tanox), or MYO-029 (Wyeth).

Another embodiment is directed to the antibody composition wherein the glycoform comprises at least four sugars.

Another embodiment is directed to a method of evaluating a biological activity of a glycopolypeptide comprising the steps of a) producing a substantially pure population of glycopolypeptides having a selected glycoform composition, and b) measuring the biological activity of the glycopolypeptide.

Another embodiment is directed to the method of paragraph [0028], wherein the glycopolypeptide is an antibody and the biological activity is (i) a binding affinity for an FcγR or (ii) antibody-dependent cell-mediated cytotoxicity.

Another embodiment is directed to the method of paragraph [0028], wherein the glycopolypeptide is an antibody and the biological activity is (i) a binding affinity for an FcγR r or (ii) enhanced phagocytosis of antibody-pathogen or antibody-antigen complexes.

Another embodiment is directed to the method of paragraph [0028], wherein the glycopolypeptide is an antibody and the biological activity is (i) a binding affinity for C1q protein or (ii) diminished complement-dependent cytotoxicity (CDC).

Another embodiment is directed to the method of paragraph [0028], wherein the glycopolypeptide is an antibody and the biological activity is (i) a binding affinity for C1q protein or (ii) enhanced complement-dependent cytotoxicity (CDC).

Another embodiment is directed to the method of paragraph [0028], wherein the glycopolypeptide is an antibody and the biological activity is (i) a binding affinity for an FcγRI and FcγRIII with a Kd of 1×10-8 M or less.

In another embodiment, the instant invention is drawn to a method of modulating complement-dependent cytotoxicity (CDC) comprising administering a glycosylation-engineered antibody.

In another embodiment, the instant invention is drawn to a method of modulating antibody dependent cellular cytotoxicity (ADCC) comprising administering a glycosylation-engineered antibody.

In another embodiment, the instant invention is drawn to a method of modulating phagocytosis of antibody-pathogen or antibody-antigen complexes comprising administering a glycosylation-engineered antibody.

Another embodiment is directed to a method of creating a generic bioequivalent of a marketed MAb by producing an antibody having the desired glycoform in a transgenic plant resulting in an antibody glycoform composition substantially more homogeneous than the glycoform composition of a marketed antibody.

Another embodiment is directed to improving the efficacy, decreasing the toxicity, and/or decreasing the dose of a marketed mAb or a mAb that has been in clinical development by introducing the preferred GNGN or G1/G2 mAb glycoform using the method of producing the antibody in a transgenic plant wherein xylosyl transferase and fucosyl transferase enzymatic activities have been substantially eliminated.

Another embodiment is directed to improving the efficacy, decreasing the toxicity, and/or decreasing the dose of a marketed mAb or a mAb that has been in clinical development by introducing the preferred GNGN or G1/G2 mAb glycoform using the method of producing the antibody in CHO wherein galactosyl transferase and/or fucosyl transferase enzymatic activities have been substantially eliminated.

Another embodiment is directed to improving the efficacy, decreasing the toxicity, and/or decreasing the dose of a marketed mAb or a mAb that has been in clinical development by introducing the preferred GNGN or G1/G2 mAb glycoform using the method of producing the antibody in yeast wherein galactosyl transferase and/or fucosyl transferase enzymatic activities have been substantially eliminated.

The present invention also describes isolated antibodies, or antigen-binding fragments thereof, that contain a substantially homogenous glycan composition for the prophylaxis and treatment of infectious diseases, inflammatory diseases and cancer.

Provided herein are antibodies or antigen-binding fragments thereof contain a sequence of amino acids set forth in any of SEQ ID NO: 1-8, where the isolated polypeptide immunospecifically binds the heavily glycosylated mucin-like domain of the Ebola virus glycoprotein. Homologs and variants of a V_{L} or a V_{H} of an antibody that specifically binds the GP of Ebola virus are typically characterized by possession of at least about 80%, for example at least about 80%, 85%, 90%, 91%, 92%, 93% 94%, 95%, 96%, 97%, 98% or 99% sequence identity counted over the full length alignment with the amino acid sequence of the antibody using the NCBI Blast 2.0, gapped blastp set to default parameters.

Provided herein are antibodies or antigen-binding fragments thereof provided herein contain a sequence of amino acids set forth in any U.S. Pat. No. 6,800,738 Ser. No. 09/705,398 filed on Nov. 2, 2000, which is hereby incorporated by reference, where the isolated polypeptide immunospecifically binds the human HER2 receptor. Homologs and variants of a V_{L} or a V_{H} of an antibody that specifically binds the human HER2 are typically characterized by possession of at least about 80%, for example at least about 80%, 85%, 90%, 91%, 92%, 93% 94%, 95%, 96%, 97%, 98% or 99% sequence identity counted over the full length alignment with the amino acid sequence of the antibody using the NCBI Blast 2.0, gapped blastp set to default parameters.

Provided herein are antibodies or antigen-binding fragments thereof provided herein contain a sequence of amino acids set forth in any U.S. Pat. No. 7,381,560 Ser. No. 09/911,692 filed on Jul. 25, 2001 and related applications, which is hereby incorporated by reference, where the isolated polypeptide immunospecifically binds the human CD20 antigen. Homologs and variants of a V_{L} or a V_{H} of an antibody that specifically binds the human CD20 are typically characterized by possession of at least about 80%, for example at least about 80%, 85%, 90%, 91%, 92%, 93% 94%, 95%, 96%, 97%, 98% or 99% sequence identity counted over the full length alignment with the amino acid sequence of the antibody using the NCBI Blast 2.0, gapped blastp set to default parameters.

Provided herein are antibodies or antigen-binding fragments thereof provided herein contain a sequence of amino acids set forth in any U.S. Pat. No. 6,818,216 Ser. No. 09/996,288 filed on Nov. 28, 2001, which is hereby incorporated by reference, where the isolated polypeptide immunospecifically binds Respiratory Syncytial Virus (RSV) antigens or antigen compositions such as fusion proteins. Homologs and variants of a V_{L} or a V_{H} of an antibody that specifically binds the antigens of the Respiratory Syncytial virus are typically characterized by possession of at least about 80%, for example at least about 80%, 85%, 90%, 91%, 92%, 93% 94%, 95%, 96%, 97%, 98% or 99% sequence identity counted over the full length alignment with the amino acid sequence of the antibody using the NCBI Blast 2.0, gapped blastp set to default parameters.

In yet another embodiment, the present invention describes deimmunized monoclonal antibodies with a highly homogeneous N-glycosylation profile carrying bi-antennary N-glycans with terminal N-acetylglucosamine on both branches (GNGN), and lacking potentially immunogenic plant specific β1,2 xylose and core α1,3 fucose. This high homogeneity is achieved through a plant manufacturing system in N. benthamiana.

In yet another embodiment, the present invention describes deimmunized monoclonal antibodies with a highly homogeneous N-glycosylation profile carrying bi-antennary N-glycans with terminal galactose on one or both branches (G1/G2) due to the presence of the galactosyl transferase enzyme and lacking potentially immunogenic plant specific 01,2 xylose and core α1,3 fucose. This high homogeneity is achieved through a plant manufacturing system in N. benthamiana.

In another embodiment, the present invention provides a composition comprising an antibody, or antigen-binding fragment thereof, with a highly homogeneous N-glycosylation profile and plant material. The plant material is selected from the group consisting of plant cell wall, plant organelle, plant cytoplasm, plant protoplast, plant cell, intact plant, viable plant, plant leaf extract, plant leaf homogenate, and chlorophyll.

These and various other advantages and novel features characterizing the present invention are also particularly pointed out in the claims attached to and forming a part of the present application. However, for a better understanding of the invention, its advantages, and objectives obtained by its use, reference should also be made to the accompanying descriptive disclosure, in which the preferred embodiments and methods of practicing the present invention are described in requisite detail.

### BRIEF DESCRIPTION OF THE DRAWINGS

FIG. 1. Sugar linkages in mammalian glycans. In the nomenclature at the top, (G2F)₂ is the same as G2F in Table 1 and (G0F)₂ is the same as G0 in Table 1;

FIG. 2 shows C1q binding ELISA. Error bars indicate standard deviation (n=3);

FIG. 3 shows a summary of dose response experiments in mice. *P<0.05 compared to 3 μg h-13F6_{CHO} and PBS (Mantel-Cox). **P=0.08 compared to 30 μg h-13F6_{CHO}; P<0.001 compared to 30 μg h-13F6_{agly} and PBS; and

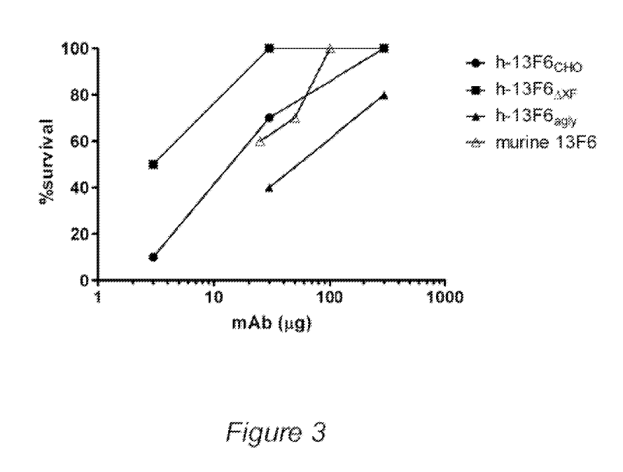

FIG. 4 depicts survival curves for the low dose (3 μg) groups of mice.

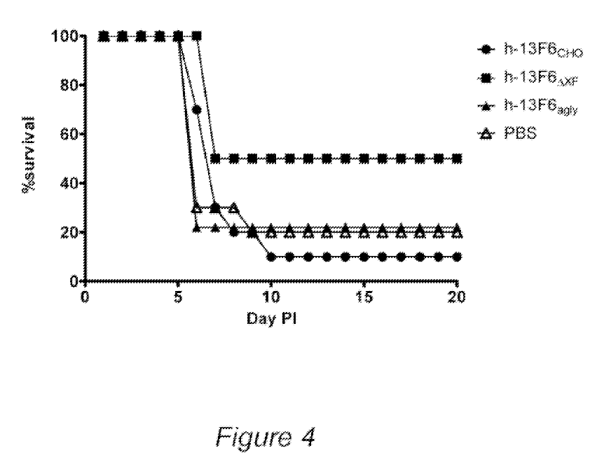

### DETAILED DESCRIPTION OF THE INVENTION

The present invention is based upon the discovery of monoclonal antibodies with substantially homogeneous glycan compositions that have enhanced binding to FcγRIIIa and FcγRI and wherein the binding to C1q can be either enhanced or reduced, as needed. These beneficial characteristics were surprisingly conferred by substantially homogeneous glycoforms, either GNGN or G1G2. Production systems for the antibody having a substantially homogeneous glycoform, GNGN or G1G2, include but are not limited to plant systems, mammalian systems and yeast systems.

Unless defined otherwise, all technical and scientific terms are used according to conventional usage and have the same meaning as is commonly understood by one of skill in the art to which this invention belongs.

In order to facilitate review of the various embodiments of this disclosure, the following explanations of specific terms are provided.

The N-glycans attached to glycoproteins differ with respect to the number of branches (antennae) comprising peripheral sugars (e.g., G1cNAc, galactose, fucose, and sialic acid) that are added to a G1cNac₂Man₃ core structure.

The term “biantennary N-glycans” refers to a complex oligosaccharide wherein the core comprises two branch terminal N-acetylglucosamine (G1cNAc), three mannose (Man) and two (G1cNAc) monosaccharide residues that are attached to the asparagine residue of the glycoprotein. The asparagine residue is generally within the conserved peptide sequence Asn-Xxx-Thr or Asn-Xxx-Ser, where Xxx is any residue except proline, aspartate, or glutamate. Subsequent glycosylation steps yield the final complex N-glycan structure. The biantennary N-glycan core structure is denoted herein as “G1cNAc₂Man₃G1cNAc₂” or GNGN.

The term “G0 glycan” is intended to mean the complex N-linked glycan having the G1cNAc₂Man₃G1cNAc₂ core structure, wherein no terminal sialic acids (NeuAcs) or terminal galactose (Gal) sugar residues are present. The G0 glycan does have a fucose residue. In plants, however, the core is substituted by a 01,2-linked xylose residue and an a1,3-linked fucose residue unlike the α1,6-linked core fucose residue found in mammals. The plant-specific β1,2-linked xylose residue and the α1,3-linked fucose residue are responsible for the immunogenicity of plant glycoproteins in humans. Thus, in one embodiment of the present invention, N. benthamiana was modified by gene knockout to eliminate expression of the endogenous plant-specific xylosyl and fucosyl transferase genes.

The term “G1 glycan” is intended to mean the complex GNGN biantennary N-glycan having the G1cNAc₂Man₃G1cNAc₂ core structure plus one terminal galactose residue.

The term “G1F glycan” is intended to mean the complex G1 biantennary N-glycan having the G1cNAc₂Man₃G1cNAc₂ core structure plus one terminal galactose residue and and one fucose residue.

The term “G2 glycan” is intended to mean the complex GNGN biantennary N-glycan having the G1cNAc₂Man₃G1cNAc₂ core structure plus two terminal galactose residues.

The term “G2F glycan” is intended to mean the complex G2 biantennary N-glycan having the G1cNAc₂Man₃G1cNAc₂ core structure plus two terminal galactose residues and one fucose residue.

The term “G1/G2 glycan” is intended to mean the complex GNGN biantennary N-glycan having the G1cNAc₂Man₃G1cNAc₂ core structure plus one or two terminal galactose residues comprising >80% galactosylated glycoforms.

The term “GN glycan” is intended to mean the complex glycan having a G1cNAc₂Man₃G1cNAc₂ core structure having only one terminal G1cNAc residue.

The term “GNF glycan” is intended to mean the complex glycan having a G1cNAc₂Man₃G1cNAc₂ core structure having only one terminal G1cNAc residue and one fucose residue.

The term “G0× glycan” is intended to mean the complex glycan having a G1cNAc₂Man₃G1cNAc₂ core structure having one xylose residue and one fucose residue.

The term “GNGNX glycan” is intended to mean the complex glycan having a G1cNAc₂Man₃G1cNAc₂ core structure having one xylose residue.

The term “GNGNF glycan” is intended to mean the complex glycan having a G1cNAc₂Man₃G1cNAc₂ core structure having two terminal G1cNAc residues and one fucose residue.

The term “GNGNFX glycan” is intended to mean the complex glycan having a G1cNAc₂Man₃G1cNAc₂ core structure having two terminal G1cNAc residues, one fucose and one xylose residue.

The term “antibody” refers to a polypeptide ligand comprising at least a light chain or heavy chain immunoglobulin variable region, or fragments thereof, which specifically recognize and bind an epitope of an antigen, such as the heavily glycosylated mucin-like domain of Ebola virus GP, the fusion glycoprotein of RSV, the cytokine TNFa, the CD20 B cell surface marker or the HER2 breast cancer marker. Antibodies are composed of a heavy chain and a light chain, each of which has a variable region, termed the variable heavy (V_{H}) region and the variable light (V_{L}) region, and a constant region. The heavy chain constant region is primarily comprised of three domains, CH1, CH2 and CH3 but may have additional components (e.g. hinge region, membrane spanning region, CH4 region). Together, the V_{H} region and the V_{L} region are responsible for binding the antigen recognized by the antibody.

The definition of antibody includes intact immunoglobulins and the variants and portions thereof well known in the art, such as Fab' fragments, F(ab)'₂ fragments, single chain Fv proteins (“scFv”), and disulfide stabilized Fv proteins (“dsFv”). A scFv protein is a fusion protein in which a light chain variable region of an immunoglobulin and a heavy chain variable region of an immunoglobulin are bound by a linker, while in dsFvs, the chains have been mutated to introduce a disulfide bond to stabilize the association of the chains.

The term antibody is used in its broadest sense and includes immunoglobulin or antibody molecules including polyclonal antibodies, hetero-conjugate antibodies, and monoclonal antibodies including murine, human, humanized and chimeric monoclonal antibodies.

Typically, a naturally occurring immunoglobulin has heavy (H) chains and light (L) chains interconnected by disulfide bonds. There are two types of light chains, lambda (λ) and kappa (κ). There are five main heavy chain classes (or isotypes) which determine the functional activity of an antibody molecule: IgM, IgD, IgG, IgA and IgE.

Each heavy and light chain contains a constant region and a variable region, (the regions are also known as “domains”). Light and heavy chain variable regions contain a “framework” region interrupted by three hypervariable regions, also called “complementarity-determining regions” or “CDRs.” The extent of the framework region and CDRs have been defined (see, Kabat et al., Sequences of Proteins of Immunological Interest, U.S. Department of Health and Human Services, 1991, which is hereby incorporated by reference). The Kabat database is now maintained online. The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found. Antibodies with different specificities (i.e. different combining sites for different antigens) have different CDRs. Although it is the CDRs that vary from antibody to antibody, only a limited number of amino acid positions within the CDRs are directly involved in antigen binding. These positions within the CDRs are called specificity determining residues (SDRs).

References to “V_{H}” or “VH” refer to the variable region of an immunoglobulin heavy chain, including that of an Fv, scFv, dsFv or Fab. References to “V_{L}” or “VL” refer to the variable region of an immunoglobulin light chain, including that of an Fv, scFv, dsFv or Fab.

A antibody or antigen binding fragment with a substantially homogenous glycan composition is comprised of glycoforms wherein 80% or greater of the glycosylated antibodies or antigen binding fragments are the GNGN glycoform in the absence of G0, G1F, G2F, GNF, GNGNF, GNGNX and GNGNFX glycoforms.

A antibody or antigen binding fragment with a substantially homogenous glycan composition is comprised of glycoforms wherein 80% or greater of the glycosylated antibodies or antigen binding fragments are the G1/G2 glycoform in the absence of G0, G1F, G2F, GNF, GNGNF, GNGNX and GNGNFX glycoforms.

A antibody or antigen binding fragment with a substantially homogenous glycan composition is comprised of glycoforms wherein 90% or greater of the glycosylated antibodies or antigen binding fragments are the GNGN glycoform in the absence of G0, G1F, G2F, GNF, GNGNF, GNGNX and GNGNFX glycoforms.

A antibody or antigen binding fragment with a substantially homogenous glycan composition is comprised of glycoforms wherein 90% or greater of the glycosylated antibodies or antigen binding fragments are the G1/G2 glycoform in the absence of G0, G1F, G2F, GNF, GNGNF, GNGNX and GNGNFX glycoforms.

A antibody or antigen binding fragment with a substantially homogenous glycan composition is comprised of glycoforms wherein 95% or greater of the glycosylated antibodies or antigen binding fragments are the GNGN glycoform in the absence of G0, G1F, G2F, GNF, GNGNF, GNGNX and GNGNFX glycoforms.

A antibody or antigen binding fragment with a substantially homogenous glycan composition is comprised of glycoforms wherein 95% or greater of the glycosylated antibodies or antigen binding fragments are the G1/G2 glycoform in the absence of G0, G1F, G2F, GNF, GNGNF, GNGNX and GNGNFX glycoforms.

A “monoclonal antibody” is an antibody produced by a single clone of B-lymphocytes or by a cell into which the light and heavy chain genes of a single antibody have been transfected. Monoclonal antibodies are produced by methods known to those of skill in the art, for instance by making hybrid antibody-forming cells from a fusion of myeloma cells with immune spleen cells. Monoclonal antibodies include murine, human, humanized and chimeric monoclonal antibodies.

A “chimeric antibody” is an antibody which comprises portions from two or more different species, such as murine and human. Most typically, chimeric antibodies include human and murine antibody domains, generally human constant regions and murine variable regions, murine CDRs and/or murine SDRs. In one embodiment of the present invention, h-13F6 comprises deimmunized murine 13F6 V_{H} and V_{L} regions joined with human IgG₁ and λ chain constant regions. This definition also includes humanized antibodies.

A “deimmunized” antibody has the immunogenic epitopes in the murine variable domains replaced with benign amino acid sequences, resulting in a deimmunized variable domain. In one embodiment of the present invention, the sequences of the T-cell epitopes located within the murine 13F6 V_{H} and V_{L} regions were identified and eliminated by introducing point mutations. As described above, the deimmunized variable domains are linked genetically to human antibody constant domains.

A “human” antibody (also called a “fully human” antibody) is an antibody that includes human framework regions and all of the CDRs from a human immunoglobulin. The variable and constant regions are derived from human germline immunoglobulin sequences. The fully human antibody may include amino acid residues introduced via random or site-specific mutagenesis in vitro or by somatic mutation in vivo. Fully human immunoglobulins can be constructed by means of genetic engineering (see for example, U.S. Pat. No. 6,090,382).

A “hybrid” antibody (also called a “chimeric” antibody) is an antibody that includes non-human framework regions and all of the CDRs from a non-human immunoglobulin. The constant regions are derived from human germline immunoglobulin sequences. The hybrid antibody may include amino acid residues introduced via random or site-specific mutagenesis in vitro or by somatic mutation in vivo.

A “humanized” immunoglobulin is an immunoglobulin including a human framework region and one or more CDRs from a non-human (for example a mouse, rat, or synthetic) immunoglobulin. The non-human immunoglobulin providing the CDRs is termed a “donor,” and the human immunoglobulin providing the framework is termed an “acceptor.” Constant regions need not be present, but if they are, they must be substantially identical to human immunoglobulin constant regions, i.e., at least about 85-90%, such as about 95% or more identical. Hence, all parts of a humanized immunoglobulin, except possibly the CDRs, are substantially identical to corresponding parts of natural human immunoglobulin sequences. A “humanized antibody” is an antibody comprising a humanized light chain and a humanized heavy chain immunoglobulin. A humanized antibody binds to the same antigen as the donor antibody that provides the CDRs. The acceptor framework of a humanized immunoglobulin or antibody may have a limited number of substitutions by amino acids taken from the donor framework. Humanized or other monoclonal antibodies can have additional conservative amino acid substitutions which have substantially no effect on antigen binding or other immunoglobulin functions. Humanized immunoglobulins can be constructed by means of genetic engineering (see for example, U.S. Pat. No. 5,585,089).

The term “binding affinity” refers to the affinity of an antibody for an antigen. In one embodiment, binding affinity is measured by an antigen/antibody dissociation rate (Kd) using surface plasmon resonance. In one example, the affinity of 13F6 for recombinant FcγRI (CD64), FcγRIII (CD 16) and C1q was determined.

In another example, the affinity of anti-CD20 mAbs (rituximab) for recombinant FcγRI (CD64), FcγRIII (CD16) and C1q was determined.

In another example, the affinity of anti-HER2 mAbs (trastuzumab) for recombinant FcγRI (CD64), FcγRIII (CD 16) and C1q was determined.

The term “conservative variants” refers to conservative amino acid substitutions that do not substantially affect or decrease the affinity of an antibody.

The term “Complementarity Determining Region (CDR)” refers to amino acid sequences which together define the binding affinity and specificity of the natural Fv region of a native Ig binding site. The light and heavy chains of an Ig each have three CDRs, designated L-CDR1, L-CDR2, L-CDR3 and H-CDR1, H-CDR2, H-CDR3, respectively. By definition, the CDRs of the light chain are bounded by the residues at positions 24 and 34 (L-CDR1), 50 and 56 (L-CDR2), 89 and 97 (L-CDR3); the CDRs of the heavy chain are bounded by the residues at positions 31 and 35b (H-CDR1), 50 and 65 (H-CDR2), 95 and 102 (H-CDR3), using the numbering convention delineated by Kabat et al., (1991) Sequences of Proteins of Immunological Interest, 5.sup.th Edition, U.S. Department of Health and Human Services, Public Health Service, National Institutes of Health, Bethesda, Md. (NIH Publication No. 91-3242).

The term “cytotoxicity” refers to the toxicity of a molecule, such as an immunotoxin, to the cells intended to be targeted, as opposed to the cells of the rest of an organism. In one embodiment, in contrast, the term “toxicity” refers to toxicity of an immunotoxin to cells other than those that are the cells intended to be targeted by the targeting moiety of the immunotoxin, and the term “animal toxicity” refers to toxicity of the immunotoxin to an animal by toxicity of the immunotoxin to cells other than those intended to be targeted by the immunotoxin.

The term “effector molecule” refers to the portion of a chimeric molecule that is intended to have a desired effect on a cell to which the chimeric molecule is targeted. Effector molecule is also known as an effector moiety (EM), therapeutic agent, or diagnostic agent, or similar terms.

The term “epitope” refers to an antigenic determinant. These are particular chemical groups or peptide sequences on a molecule that are antigenic, i.e. that elicit a specific immune response. An antibody specifically binds a particular antigenic epitope on a polypeptide.

The term “immune response” refers to a response of a cell of the immune system, such as a B cell, T cell, or monocyte, to a stimulus. In one embodiment, the response is specific for a particular antigen, the heavily glycosylated mucin-like domain of Ebola virus GP (an “antigen-specific response”).

The term “Natural Killer (NK) cells” refers to a form of lymphocyte that kills a target cell through antibody-dependent cell-mediated cytotoxicity. NK cells express the surface receptor FcγRIII (CD16).

As used herein, the term “nucleic acid,” “nucleic acid sequence,” “polynucleotide,” or similar terms, refers to a deoxyribonucleotide or ribonucleotide, oligonucleotide or polynucleotide, including single- or double-stranded forms, and coding or non-coding (e.g., “antisense”) forms. The term encompasses nucleic acids containing known analogues of natural nucleotides. The term also encompasses nucleic acids including modified or substituted bases as long as the modified or substituted bases interfere neither with the Watson-Crick binding of complementary nucleotides or with the binding of the nucleotide sequence by proteins that bind specifically, such as zinc finger proteins. The term also encompasses nucleic-acid-like structures with synthetic backbones. DNA backbone analogues provided by the invention include phosphodiester, phosphorothioate, phosphorodithioate, methylphosphonate, phosphoramidate, alkyl phosphotriester, sulfamate, 3′-thioacetal, methylene(methylimino), 3′-N-carbamate, morpholino carbamate, and peptide nucleic acids (PNAs); see Oligonucleotides and Analogues, a Practical Approach, edited by F. Eckstein, IRL Press at Oxford University Press (1991); Antisense Strategies, Annals of the New York Academy of Sciences, Volume 600, Eds. Baserga and Denhardt (NYAS 1992); Milligan (1993) J. Med. Chem. 36:1923-1937; Antisense Research and Applications (1993, CRC Press). PNAs contain non-ionic backbones, such as N-(2-aminoethyl) glycine units. Phosphorothioate linkages are described, e.g., by U.S. Pat. Nos. 6,031,092; 6,001,982; 5,684,148; see also, WO 97/03211; WO 96/39154; Mata (1997) Toxicol. Appl. Pharmacol. 144:189-197. Other synthetic backbones encompassed by the term include methylphosphonate linkages or alternating methylphosphonate and phosphodiester linkages (see, e.g., U.S. Pat. No. 5,962,674; Strauss-Soukup (1997) Biochemistry 36:8692-8698), and benzylphosphonate linkages (see, e.g., U.S. Pat. No. 5,532,226; Samstag (1996) Antisense Nucleic Acid Drug Dev 6:153-156). Such analogues can be employed in the preparation and use of antisense nucleic acids as is well known in the art, such as for the purpose of inhibiting transcription. Additionally, the recitation of a nucleic acid sequence includes its complement unless the complement is specifically excluded or the context makes it clear that only one strand of the nucleic acid sequence is intended to be utilized. Additionally, the recitation of a nucleic acid sequence includes DNA, RNA, or DNA-RNA hybrids unless the context makes it clear that only one specific form of the nucleic acid sequence is intended to be utilized.

As used herein, the amino acids, which occur in the various amino acid sequences appearing herein, are identified according to their well-known, three-letter or one-letter abbreviations. The nucleotides, which occur in the various DNA fragments, are designated with the standard single-letter designations used routinely in the art.

In a peptide or protein, suitable conservative substitutions of amino acids are known to those of skill in this art and may be made generally without altering the biological activity of the resulting molecule. Those of skill in this art recognize that, in general, single amino acid substitutions in non-essential regions of a polypeptide do not substantially alter biological activity (see, e.g. J. D. Watson et al,. “Molecular Biology of the Gene” (4th Edition, 1987, Benjamin/Cummings, Palo Alto), p. 224). Specifically, in particular, the conservative amino acid substitutions can be any of the following: (1) any of isoleucine for leucine or valine, leucine for isoleucine, and valine for leucine or isoleucine; (2) aspartic acid for glutamic acid and glutamic acid for aspartic acid; (3) glutamine for asparagine and asparagine for glutamine; and (4) serine for threonine and threonine for serine. Other substitutions can also be considered conservative, depending upon the environment of the particular amino acid. For example, glycine (G) and alanine (A) can frequently be interchangeable, as can be alanine and valine (V). Methionine (M), which is relatively hydrophobic, can frequently be interchanged with leucine and isoleucine, and sometimes with valine. Lysine (K) and arginine (R) are frequently interchangeable in locations in which the significant feature of the amino acid residue is its charge and the different pK's of these two amino acid residues or their different sizes are not significant. Still other changes can be considered “conservative” in particular environments. For example, if an amino acid on the surface of a protein is not involved in a hydrogen bond or salt bridge interaction with another molecule, such as another protein subunit or a ligand bound by the protein, negatively charged amino acids such as glutamic acid and aspartic acid can be substituted for by positively charged amino acids such as lysine or arginine and vice versa. Histidine (H), which is more weakly basic than arginine or lysine, and is partially charged at neutral pH, can sometimes be substituted for these more basic amino acids. Additionally, the amides glutamine (Q) and asparagine (N) can sometimes be substituted for their carboxylic acid homologues, glutamic acid and aspartic acid.

The present invention contemplates peptide modifications: the polypeptides of the present invention include synthetic embodiments of peptides described herein. In addition, analogs (non-peptide organic molecules), derivatives (chemically functionalized peptide molecules obtained starting with the disclosed peptide sequences) and variants (homologs) of these proteins can be utilized in the methods described herein. Each polypeptide is comprised of a sequence of amino acids, which may be either L- and/or D-amino acids, naturally occurring and otherwise.

Homologs and variants of V_{L} or V_{H} of antibodies that specifically binds the GP of Ebola virus, or CD20, or HER2 are typically characterized by possession of at least about 80%, for example at least about 80%, 85%, 90%, 91%, 92%, 93% 94%, 95%, 96%, 97%, 98% or 99% sequence identity counted over the full length alignment with the amino acid sequence of the antibody using the NCBI Blast 2.0, gapped blastp set to default parameters. For comparisons of amino acid sequences of greater than about 30 amino acids, the Blast 2 sequences function is employed using the default BLOSUM62 matrix set to default parameters, (gap existence cost of 11, and a per residue gap cost of 1). When aligning short peptides (fewer than around 30 amino acids), the alignment should be performed using the Blast 2 sequences function, employing the PAM30 matrix set to default parameters (open gap 9, extension gap 1 penalties). Proteins with even greater similarity to the reference sequences will show increasing percentage identities when assessed by this method, such as at least 80%, at least 85%, at least 90%, at least 95%, at least 98%, or at least 99% sequence identity. When less than the entire sequence is being compared for sequence identity, homologs and variants will typically possess at least 80% sequence identity over short windows of 10-20 amino acids, and may possess sequence identities of at least 85% or at least 90% or 95% depending on their similarity to the reference sequence. Methods for determining sequence identity over such short windows are available at the NCBI website on the internet. One of skill in the art will appreciate that these sequence identity ranges are provided for guidance only; it is entirely possible that strongly significant homologs could be obtained that fall outside of the ranges provided.

Thus, when the monoclonal antibodies as disclosed herein are used to prevent or treat disease, the heavy chain may be used alone, or both heavy and light chains together may be present. The invention also contemplates monoclonal antibodies having sequences that are at least 80%, preferably 90%, and more preferably 95% homologous to the heavy and/or light chain regions described in U.S. Pat. No. 6,800,738 Ser. No. 09/705,398 filed on Nov. 2, 2000, U.S. Pat. No. 7,381,560 Ser. No. 09/911,692 filed on Jul. 25, 2001, U.S. Pat. No. 6,818,216 Ser. No. 09/996,288 filed on Nov. 28, 2001, and SEQ ID NO:1, SEQ ID NO:2, SEQ ID NO:3, SEQ ID NO:4, SEQ ID NO:5, SEQ ID NO:6, SEQ ID NO:7 and SEQ ID NO:8 and which compete for binding Ebola GP. As noted above, there can be a 5% variation normally in even the more conserved framework regions, and someone having ordinary skill in this art using known techniques would be able to determine without undue experimentation such homologous, competing monoclonal antibodies. The invention also contemplates monoclonal antibodies that compete with h-13F6 for binding to Ebola GP, and which have the herein described CDRs in the appropriate positions as determined by the Kabat system in the light and/or heavy chains.

As used herein, “expression vector” refers to a plasmid, virus or other vehicle known in the art that has been manipulated by insertion or incorporation of heterologous DNA, such as nucleic acid encoding the fusion proteins herein or expression cassettes provided herein. Such expression vectors contain a promoter sequence for efficient transcription of the inserted nucleic acid in a cell. The expression vector typically contains an origin of replication, and a promoter, as well as specific genes that permit phenotypic selection of transformed cells.

As used herein, “host cells” are cells in which a vector can be propagated and its DNA expressed. The term also includes any progeny of the subject host cell. It is understood that all progeny may not be identical to the parental cell since there may be mutations that occur during replication. Such progeny are included when the term “host cell” is used. Methods of stable transfer where the foreign DNA is continuously maintained in the host are known in the art.

As used herein, an expression or delivery vector refers to any plasmid or virus into which a foreign or heterologous DNA may be inserted for expression in a suitable host cell—i.e., the protein or polypeptide encoded by the DNA is synthesized in the host cell's system. Vectors capable of directing the expression of DNA segments (genes) encoding one or more proteins are referred to herein as “expression vectors”. Also included are vectors that allow cloning of cDNA (complementary DNA) from mRNAs produced using reverse transcriptase. As used herein, a gene refers to a nucleic acid molecule whose nucleotide sequence encodes an RNA or polypeptide. A gene can be either RNA or DNA. Genes may include regions preceding and following the coding region (leader and trailer) as well as intervening sequences (introns) between individual coding segments (exons).

As used herein, “isolated,” with reference to a nucleic acid molecule or polypeptide or other biomolecule means that the nucleic acid or polypeptide has separated from the genetic environment from which the polypeptide or nucleic acid were obtained. It may also mean altered from the natural state. For example, a polynucleotide or a polypeptide naturally present in a living animal is not “isolated”, but the same polynucleotide or polypeptide separated from the coexisting materials of its natural state is “isolated”, as the term is employed herein. Thus, a polypeptide or polynucleotide produced and/or contained within a recombinant host cell is considered isolated. Also intended as an “isolated polypeptide” or an “isolated polynucleotide” are polypeptides or polynucleotides that have been purified, partially or substantially, from a recombinant host cell or from a native source. For example, a recombinantly produced version of a compound can be substantially purified by the one-step method described in Smith et al. (1988) Gene 67:3140. The terms isolated and purified are sometimes used interchangeably.

Thus, by “isolated” the nucleic acid is free of the coding sequences of those genes that, in a naturally-occurring genome immediately flank the gene encoding the nucleic acid of interest. Isolated DNA may be single-stranded or double-stranded, and may be genomic DNA, cDNA, recombinant hybrid DNA, or synthetic DNA. It may be identical to a native DNA sequence, or may differ from such sequence by the deletion, addition, or substitution of one or more nucleotides.

Isolated or purified as it refers to preparations made from biological cells or hosts means any cell extract containing the indicated DNA or protein, including a crude extract of the DNA or protein of interest. For example, in the case of a protein, a purified preparation can be obtained following an individual technique or a series of preparative or biochemical techniques and the DNA or protein of interest can be present at various degrees of purity in these preparations. The procedures may include for example, but are not limited to, ammonium sulfate fractionation, gel filtration, ion exchange chromatography, affinity chromatography, density gradient centrifugation, electrophoresis, electrofocusing, chromatofocusing, or other protein purification techniques known in the art.

A preparation of DNA or protein that is “substantially pure” or “isolated” should be understood to mean a preparation free from naturally occurring materials with which such DNA or protein is normally associated in nature. “Essentially pure” should be understood to mean a “highly” purified preparation that contains at least 95% of the DNA or protein of interest.

As used herein, the term “vector” refers to a nucleic acid molecule capable of transporting between different genetic environments another nucleic acid to which it has been operatively linked. Preferred vectors are those capable of autonomous replication and expression of structural gene products present in the DNA segments to which they are operatively linked. Vectors, therefore, preferably contain the replicons and selectable markers described earlier.

### GNGN and G1/G2 Antibodies with Altered Fcγ Receptor and C1q Binding

All publications, including but not limited to patents and patent applications, cited in this specification are herein incorporated by reference as though fully set forth in the present application.

While the preferred embodiments of the present invention are illustrated below in numerical order, it is to be understood that the invention is not limited to the precise instructions and embodiments disclosed herein and that the right to all modifications coming within the scope of the following claims is reserved.

Disclosed herein are monoclonal antibodies that possess altered Fcγ receptor and C1q binding characteristics. The present invention discloses novel glycoforms produced in plant, mammalian cell, and yeast manufacturing systems to generate mAbs with said altered Fcγreceptor or C1q binding. It is contemplated by this disclosure that certain novel aspects of the present invention can be practiced with many modified mAbs to generate both humanized and fully human mAbs. For example, humanized and fully human mAbs may be generated using techniques well known in the art in combination with the teachings of the present invention to produce glycan-optimized mAbs as therapeutic or preventive drugs. CDR grafted or humanized mAbs are well known in the art and can be generated according to Winter and Harris, Immunol. Today 14:243-246, 1993. Fully human immunoglobulins can be constructed by means of genetic engineering (see for example, U.S. Pat. No. 6,090,382 and U.S. Pat. No. 7,824,681).

A preferred embodiment of the present invention is an isolated monoclonal antibody, or antigen binding fragment thereof, as an immunoprotectant for Ebola virus. The mAb comprises a heavy chain variable region and a light chain variable region, wherein the heavy chain variable region comprises the amino acid sequence as set forth in SEQ ID NO. 1 and wherein the light chain variable region comprises the amino acid sequence as set forth in SEQ ID NO. 2. The light chain variable region further comprises a CDR1 domain comprising the amino acid sequence as set forth in SEQ ID NO. 3; a CDR2 domain comprising the amino acid sequence as set forth in SEQ ID NO. 4; and a CDR3 domain comprising the amino acid sequence as set forth in SEQ ID NO. 5. The heavy chain variable region further comprises a CDR1 domain comprising the amino acid sequence as set forth in SEQ ID NO. 6; a CDR2 domain comprising the amino acid sequence as set forth in SEQ ID NO. 7; and a CDR3 domain comprising the amino acid sequence as set forth in SEQ ID NO. 8.

A preferred embodiment of the present invention is an isolated monoclonal antibody, or antigen binding fragment thereof, containing a substantially homogeneous glycan without sialic acid, galactose, or fucose. The monoclonal antibody comprises a heavy chain variable region and a light chain variable region, both of which may be attached to heavy chain or light chain constant regions respectively. The aforementioned substantially homogeneous glycan is covalently attached to the heavy chain constant region.

A preferred embodiment of the present invention is an isolated monoclonal antibody, or antigen binding fragment thereof, containing a substantially homogeneous glycan without sialic acid or fucose. The monoclonal antibody comprises a heavy chain variable region and a light chain variable region, both of which may be attached to heavy chain or light chain constant regions respectively. The aforementioned substantially homogeneous glycan is covalently attached to the heavy chain constant region.

Another embodiment of the present invention comprises a mAb with a novel Fc glycosylation pattern. The isolated monoclonal antibody, or antigen binding fragment thereof, is present in a substantially homogenous composition represented by the GNGN or G1/G2 glycoform. Fc glycosylation plays a significant role in anti-viral and anti-cancer properties of therapeutic mAbs. The disclosure of the present invention are in line with a recent study that shows increased anti-lentivirus cell-mediated viral inhibition of a fucose free anti-HIV mAb in vitro. This embodiment of the present invention with homogenous glycans lacking a core fucose, showed increased protection against specific viruses by a factor greater than two-fold. Elimination of core fucose dramatically improves the ADCC activity of mAbs mediated by natural killer (NK) cells, but appears to have the opposite effect on the ADCC activity of polymorphonuclear cells (PMNs).

The isolated monoclonal antibody, or antigen binding fragment thereof, comprising a substantially homogenous composition represented by the GNGN or G1/G2 glycoform exhibits increased binding affinity for FcγRI and FcγRIII compared to the same antibody without the substantially homogeneous GNGN glycoform and with G0, G1F, G2F, GNF, GNGNF or GNGNFX containing glycoforms. In one embodiment of the present invention, the antibody dissociates from FcγRI with a Kd of 1×10⁻⁸ M or less and and from FcγRIII with a Kd of 1×10⁻⁷ M or less.

The monoclonal antibodies of the present invention recognize and bind to an epitope of the Ebola virus GP corresponding to the amino acid sequence of SEQ ID NO. 9.

Nucleic acids encoding the amino acid sequences of the monoclonal antibody, or antigen binding fragments thereof are also provided herein. Examples of the nucleic acid sequences are represented in SEQ ID NOs. 10-15.

Without limiting the scope of the present invention, three manufacturing systems have been disclosed herein for the production of the monoclonal antibodies of the present invention namely in CHO cells, yeast cells, and in N. benthamiana. The nucleotide sequence for the heavy and light chain variable and constant regions were codon optimized for use in each expression system. The nucleic acid encoding the heavy chain variable region of the monoclonal antibody, or antigen binding fragment thereof, for use in the CHO and yeast expression systems comprises the nucleic acid sequence of SEQ ID NO. 10. The nucleic acid encoding the heavy chain constant region of the monoclonal antibody, or antigen binding fragment thereof, for use in the CHO and yeast expression systems comprises the nucleic acid sequence of SEQ ID NO. 11. The nucleic acid encoding the light chain variable and constant regions of the monoclonal antibody, or antigen binding fragment thereof, for use in the CHO and yeast expression systems comprises the nucleic acid sequence of SEQ ID NO. 12.

The nucleic acid encoding the heavy chain variable region of the monoclonal antibody, or antigen binding fragment thereof, for use in the N. benthamiana expression system comprises the nucleic acid sequence of SEQ ID NO. 13. The nucleic acid encoding the heavy chain constant region of the monoclonal antibody, or antigen binding fragment thereof, for use in the N. benthamiana expression system comprises the nucleic acid sequence of SEQ ID NO. 14. The nucleic acid encoding the light chain variable and constant regions of the monoclonal antibody, or antigen binding fragment thereof, for use in the N. benthamiana expression system comprises the nucleic acid sequence of SEQ ID NO. 15.

The antigen binding fragment of the present invention may be a Fab' fragment, a F(ab)'2 fragment, or a scFv fragment.

The variable regions have been deimmunized by introducing point mutations to remove human T-cell epitopes. The deimmunized variable regions were then chimerized with human IgGᵢ constant regions to generate an embodiment of the present invention safe for human use. The amino acid sequence for the human IgG₁ constant region is given in SEQ ID NO. 16. The heavy chain constant region is not limited to the IgG isotype but may be any of the following IgA, IgD, IgE, IgG, and IgM but is prefereably IgG and more preferably IgGᵢ. In addition, the light chain region comprises a rare VXx light chain variable region that may have a conformational affect on the Fc region. The crystal structure for the murine parental mAb shows that the three light-chain CDRs adopt unusual conformations distinct from Vι and other Vλ light chains (19). This unique feature may be one possible explanation for an enhanced affinity of this embodiment of the mAb for FcγRIIIa. The amino acid sequence for the Xx light chain variable and constant regions are given in SEQ ID NOs. 2 and 17 respectively.

The monoclonal antibodies of the present invention recognize and bind to an epitope of the Ebola virus GP corresponding to the amino acid sequence of SEQ ID NO. 9.

Nucleic acids encoding the amino acid sequences of the monoclonal antibody, or antigen binding fragments thereof are also provided herein. Examples of the nucleic acid sequences are represented in SEQ ID NOs. 10-15.

Without limiting the scope of the present invention, different manufacturing systems have been disclosed herein for the production of the monoclonal antibodies of the present invention. The nucleotide sequence for the heavy and light chain variable and constant regions were codon optimized for use in each expression system. The nucleic acid encoding the heavy chain variable region of the monoclonal antibody, or antigen binding fragment thereof, for use in the CHO and yeast expression systems comprises the nucleic acid sequence of SEQ ID NO. 10. The nucleic acid encoding the heavy chain constant region of the monoclonal antibody, or antigen binding fragment thereof, for use in the CHO and yeast expression system comprises the nucleic acid sequence of SEQ ID NO. 11. The nucleic acid encoding the light chain variable and constant regions of the monoclonal antibody, or antigen binding fragment thereof, for use in the CHO and yeast expression system comprises the nucleic acid sequence of SEQ ID NO. 12.

The nucleic acid encoding the heavy chain variable region of the monoclonal antibody, or antigen binding fragment thereof, for use in the N. benthamiana expression system comprises the nucleic acid sequence of SEQ ID NO. 13. The nucleic acid encoding the heavy chain constant region of the monoclonal antibody, or antigen binding fragment thereof, for use in the N. benthamiana expression system comprises the nucleic acid sequence of SEQ ID NO. 14. The nucleic acid encoding the light chain variable and constant regions of the monoclonal antibody, or antigen binding fragment thereof, for use in the N. benthamiana expression system comprises the nucleic acid sequence of SEQ ID NO. 15.

Without limiting the scope of the present invention, three manufacturing systems have been disclosed herein for the production of the monoclonal antibodies of the present invention namely in CHO cells, plants and yeast cells. The nucleotide sequence for the heavy and light chain variable and constant regions were codon optimized for use in each expression system.

Nucleotide molecules encoding the mAbs can be readily produced by one of skill in the art using the amino acid sequences provided herein and the genetic code. One of skill in the art can construct a variety of clones containing functionally equivalent nucleic acids, such as nucleic acids which differ in sequence but which encode the same antibody sequence.

Nucleic acids can be prepared by amplification methods including polymerase chain reaction (PCR). In addition, a wide variety of cloning methods, host cells and in vitro amplification methodologies are well known to persons of skill in the art.

In one embodiment of the present invention the host cell is a plant cell more specifically a plant cell from N. benthamiana. The plant cell has been modified by RNAi or gene knockout to eliminate expression of plant-specific xylosyl as well as plant specific-fucosyl transferase genes. Using techniques known in the art, the mAbs of the present invention can be produced in any production system suitable for producing the desired prophylactic and therapeutic effects of the present invention.

For example, another embodiment of the present invention includes a manufacturing system using traditional mammalian cell culture production in Chinese Hamster Ovary (CHO) cells. Depending on the desired glycosylation patterns, the disclosure of the present invention contemplates the use of different manufacturing systems generating a variety of Fc glycosylation patterns. In the preferred embodiment, the desired glycosylation pattern is the GNGN glycoform.

For example, another embodiment of the present invention includes a manufacturing system using traditional mammalian cell culture production in Chinese Hamster Ovary (CHO) cells. Depending on the desired glycosylation patterns, the disclosure of the present invention contemplates the use of different manufacturing systems generating a variety of Fc glycosylation patterns. In the preferred embodiment, the desired glycosylation pattern is the G1G2 glycoform.

In a further embodiment of the present invention includes a manufacturing system using yeast cells. Depending on the desired glycosylation patterns, the disclosure of the present invention contemplates the use of different manufacturing systems generating a variety of Fc glycosylation patterns. In the preferred embodiment, the desired glycosylation pattern is the GNGN or G1G2 glycoform.

Another embodiment of the present invention includes a pharmaceutical composition comprising the antibody, or antigen-binding fragment thereof, of the present invention and a pharmaceutically acceptable carrier.

Pharmaceutical compositions according to the present invention can be formulated for mucosal administration or for parenteral administration. The route of administration depends on the chemical nature of the active species, the condition of the patient, and pharmacokinetic considerations such as liver or kidney function.

Another embodiment of the present invention includes the pharmaceutical use of the substantially homogeneous GNGN or G1/G2 antibody glycoform for any prophylactic of therpeutic treatment where enhanced ADCC, enhanced phagocytosis, and reduced CDC, embodied in the same antibody glycoform, would be of medical benefit.

A further embodiment of the present invention includes the pharmaceutical use of the substantially homogeneous GNGN or G1/G2 antibody glycoform for any prophylactic of therpeutic treatment where enhanced ADCC and enhanced phagocytosis, embodied in the same antibody glycoform, would be of medical benefit.

A further embodiment of the present invention includes the pharmaceutical use of the substantially homogeneous GNGN or G1/G2 antibody glycoform for any prophylactic of therpeutic treatment where enhanced ADCC and reduced CDC, embodied in the same antibody glycoform, would be of medical benefit.

A further embodiment of the present invention includes the pharmaceutical use of the substantially homogeneous GNGN or G1/G2 antibody glycoform for any prophylactic of therpeutic treatment where enhanced phagocytosis and reduced CDC, embodied in the same antibody glycoform, would be of medical benefit.

In another therapeutic approach, the mAbs of the present invention may be used in the active immunization of a patient using an anti-idiotypic antibody raised against one of the present monoclonal antibodies. Immunization with an anti-idiotype which mimics the structure of the epitope could elicit an active anti-antigen response (Linthicum, D. S. and Farid, N. R., Anti-Idiotypes, Receptors, and Molecular Mimicry (1988), pp 1-5 and 285-300).

Another embodiment of the present invention is a method of treating a subject afflicted with a viral infection comprising administering to the subject a therapeutically effective amount of the pharmaceutical composition comprising the monoclonal antibody, or antigen binding fragment thereof, of the present invention, wherein the antibody, or antigen binding fragment thereof, recognizes and binds the virus.

Another embodiment of the present invention is a method of treating a subject afflicted with a viral infection comprising administering to the subject a prophylactically effective amount of the pharmaceutical composition comprising the monoclonal antibody, or antigen binding fragment thereof, of the present invention, wherein the antibody, or antigen binding fragment thereof, recognizes and binds the virus.

Another embodiment of the present invention is a method of treating a subject afflicted with cancer comprising administering to the subject a therapeutically effective amount of the pharmaceutical composition comprising the monoclonal antibody, or antigen binding fragment thereof, of the present invention, wherein the antibody, or antigen binding fragment thereof, recognizes and binds the cancerous cells.

Another embodiment of the present invention is a method of treating a subject afflicted with an autoimmune disease comprising administering to the subject a therapeutically effective amount of the pharmaceutical composition comprising the monoclonal antibody, or antigen binding fragment thereof, of the present invention, wherein the antibody, or antigen binding fragment thereof, recognizes and binds the auto-antigen.

Another embodiment of the present invention is a method of treating a subject afflicted with an inflammatory disease comprising administering to the subject a therapeutically effective amount of the pharmaceutical composition comprising the monoclonal antibody, or antigen binding fragment thereof, of the present invention, wherein the antibody, or antigen binding fragment thereof, recognizes and binds the antigen causing inflammation.

Furthermore, it is understood by those skilled in the art that the compounds of the present invention, including but not limited to pharmaceutical compositions and formulations containing these compounds can be used in a wide variety of combination therapies to treat the conditions and diseases described above. As described above, all compounds within the scope of the present invention can be used to formulate appropriate pharmaceutical compositions, and such pharmaceutical compositions can be used to treat the conditions described above.

### Pharmaceutical Formulation and Administration

Toxicity and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit large therapeutic indices are preferred.

The data obtained from these cell culture assays and animal studies can be used in formulating a range of dosages for use in humans. The dosage of such compounds lies preferably within a range of circulating concentrations that include the ED50 with little or no toxicity. The dosage may vary within this range depending upon the dosage form employed and the route of administration utilized.

The exact formulation, route of administration and dosage can be chosen by the individual physician in view of the mammal's condition. (See e.g. Fingl et al., in The Pharmacological Basis of Therapeutics, 1975, Ch. 1 p. 1). It should be noted that the attending physician would know how to and when to terminate, interrupt, or adjust administration due to toxicity, or to organ dysfunctions.

Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency will also vary according to the age, body weight, and response of the individual mammal.

Depending on the specific conditions being treated, such agents may be formulated and administered systemically or locally. Techniques for formulation and administration may be found in Remington's Pharmaceutical Sciences, 18th ed., Mack Publishing Co., Easton, Pa. (1990), which is incorporated herein by reference.

For injection, the agents of the invention may be formulated in aqueous solutions, preferably in physiologically compatible buffers such as Hanks's solution, Ringer's solution, or physiological saline buffer.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection.

Pharmaceutical compositions suitable for use in the present invention include compositions wherein the active ingredients are contained in an effective amount to achieve its intended purpose. Determination of the effective amounts is well within the capability of those skilled in the art, especially in light of the detailed disclosure provided herein. In addition to the active ingredients, these pharmaceutical compositions may contain suitable pharmaceutically acceptable carriers comprising excipients and auxiliaries which facilitate processing of the active compounds into preparations which can be used pharmaceutically. The pharmaceutical compositions of the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration include aqueous solutions of the active compounds in water-soluble form. Additionally, suspensions of the active compounds may be prepared as appropriate oily injection suspensions.

Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

### EXAMPLES

### Experimental Procedures

N. benthamiana expression vectors - Heavy and light chain variable regions joined with human constant regions were first codon optimized for expression in Nicotiana benthamiana. An aglycosylated mAb was designed by mutating the heavy chain constant region N-glycosylaton site (N297A). Genes were synthesized (GeneArt, AG) and subsequently cloned into plant (TMV and PVX) expression vectors (Icon Genetics, GmbH [34,35]), followed by transformation into Agrobacterium tumefaciens strain ICF320.

Production of mAbs in N. benthamiana—For transient expression of mAb genes in planta, we used the “magnifection” procedure (Icon Genetics, Halle (Saale), Germany) as described [34,35], with minor modifications. Plants grown for 4 weeks in an enclosed growth culture room with 20-23° C. were used for vacuum infiltration. Equal volumes of overnight-grown Agrobacterium cultures were mixed in the infiltration buffer 10 mM MES pH 5.5 and 10 mM MgSO4 resulting in a 1:1000 dilution for each individual culture. The infiltration solution was transferred into a 20 L custom built (Kentucky Bioprocessing, Owensboro, Ky.) vacuum chamber. The aerial parts of entire plants were dipped upside down into the bacterial/buffer solution. A vacuum of 0.5 bars was applied for 2 min. Post infiltration, plants were returned to the growth room under standard growing conditions. Eight days post-infiltration, the leaf tissue was extracted in a juicer (Green Star, Model GS-1000), using 25 ml of chilled extraction buffer (100mM Tris, 40 mM ascorbic acid, 1mM EDTA) per 100 g of green leaf tissue. The plant-derived extract was clarified by lowering the pH of the extract to pH 4.8 with 1 M phosphoric acid then re-adjusting it to pH 7.5 with 2 M Tris base to insolubilize plant debris, followed by centrifugation at 16,000 ×g for 30 min. The supernatant was transferred and re-centrifuged at 16,000 ×g for an additional 30 min. The clarified extract was filtered through 0.2 μm prior to concentration via Minim Tangential Flow Filtration System (Pall) then 0.2 μm filtered again before loading onto 5 ml HiTrap MabSelect SuRe (GE Healthcare) Protein A column at 2 ml/min. The column then was washed with running buffer (50 mM HEPES/100 mM NaC1, pH 7.5) and eluted with 0.1 M acetic acid, pH 3.0. The resulting eluate was neutralized to pH 7 using 2 M Tris, pH 9.0 and supplemented with Tween 80 to 0.01%. The mAb solution was then polished via Q filtration (Mustang Acrodisc Q membrane; Pall), aliquoted and stored at −80° C. until used.

ΔXTFT Plants used for production of mAbs devoid of xylose and fucose.

Transcription activator-like effector (TALE) proteins, a large group of bacterial plant pathogen proteins, are used to knock out the xylosyl and fucosyl transferase genes as described [36]. In brief, TALE proteins contain a varying number of centrally located tandem 34-amino-acid repeats that mediate binding to a specific DNA target sequence, referred to as the effector binding elements (EBE). Each repeat is nearly identical except for two variable amino acids at positions 12 and 13, known as repeat variable diresidues (RVD). Polymorphism in the number of repeats (a range of 13-33) and in the RVD composition collectively determines the DNA binding specificity of individual TALE proteins. Remarkably, recognition of a specific DNA sequence is based on a fairly simple code wherein one base of the DNA target site is recognized by the RVD of one repeat (i.e. one repeat/one nucleotide).
The sequential repeat arrangement in a single TALE protein thus specifies the contiguous DNA sequence that will be bound by that TALE protein and the adjacent DNA of the target gene can be inactivated by the Fokl nuclease that has been covalently attached to the C-terminus of the TALE protein.

Production of mAbs from CHO cells. A stable mAb-expressing CHO cell line, either wild type, lec8 (galactose deficient [48]) or lecl3 (fucose deficient [49]) mutant strain was cultured in CD OptiCHO medium (Invitrogen) and supplemented daily with CHO Feed Bioreactor Supplement (Sigma). The CHO culture was grown in suspension using 37° C. shaker with glucose level manually monitored daily and adjusted with sterile 45% Glucose Solution (Mediatech). The culture was terminated when cell viability reached below 20%. The conditioned medium was harvested and clarified via centrifugation. The clarified conditioned medium was filtered (0.2 μm) prior to concentration via Minim Tangential Flow Filtration System (Pall). The conditioned medium was concentrated 10-fold, filtered (0.2 μm), and loaded onto 1 ml HiTrap MabSelect SuRe (GE Healthcare) Protein A column at 0.5 ml/min. The column then was washed with 1× PBS running buffer and eluted with 0.1 M acetic acid, pH 3.0. The resulting eluate was neutralized to pH 7 using 2 M Tris, pH 9.0 and buffer-exchanged against 1× PBS with 0.01% Tween 80 using Amicon Ultra (Millipore). The mAb solution was then polished via Q filtration (Sartobind Q; Sartorius), aliquoted and stored at −80° C. until used. All purified mAb variants (CHO, AXF plant, yeast, agly) were fully assembled as determined by SDS-PAGE and had less than 5% aggregate as determined by HPLC-SEC.

Production of mAbs in yeast. Fermentation conditions: the primary culture was prepared by inoculating a 1-L baffled flask containing 200 ml of BMGY media with 10 ml of a seed culture. The cells from the primary culture were transferred to inoculate the fermenter. The fermentation medium contained: 40 g glycerol; 15 g sorbitol; 2.3 g K₂HPO₄; 11.9 g KH₂PO₄; 10 g yeast extract; 20 g peptone; 1 g casein amino acids; 4×10⁻³ g biotin; 13.4 g YNB; per liter of medium. Fermentations were conducted in 3 L (1.5 L initial volume) dished-bottom Applikon bioreactors. The fermenters were run in fed-batch mode under the following conditions: the temperature was set at 24° C. and the pH was adjusted to 6.5 with NH₄OH. The dissolved oxygen (DO) was maintained at 20% by adjusting agitation rate (450-1,000 r.p.m.) and addition of pure oxygen. The airflow rate was maintained at 0.5 vvm. After depletion of the initial glycerol (40 g/L) a 50% glycerol solution containing 12 ml/L PTM1 salts was fed at an average rate of 8 ml/L/h until the desired biomass of 250 g _{wcw}/L was reached. After a 30 min starvation period the methanol feed (100% methanol with 12 ml/L PTM1 salts) was initiated. An exponential feeding rate beginning with 3 g/L/h and increasing at a specific rate of 0.01 1/h was continued for 30 to 40 h. After the fermentation the supernatant was obtained by centrifugation and used for further purification of the antibody.
Antibody purification: the antibody was captured by affinity chromatography from the supernatant medium of P. pastoris fermentations using a Streamline rProtein A resin from GE Healthcare. The resin was equilibrated with 50 mM Tris-HC1 pH 7 and the supernatant medium was adjusted at the same pH. The column was washed with 4 column volumes of the same buffer and the antibody was eluted with 100 mM Glycine-HC1 pH 3. The eluted protein was neutralized immediately with 1 M Tris-HC1, pH 7. A phenyl sepharose fast flow resin (GE Healthcare) was used as a second purification step. The column was equilibrated in 20 mM Tris-HC1 pH 7, 1 M (NH₄)₂SO₄ and the sample obtained from the first column was applied to the phenyl sepharose column after adding (NH₄)₂SO₄ to a final concentration of 1 M. The elution was performed by developing a gradient over 10 column volumes ranging from 1 M to 0 M (NH₄)₂SO₄ in 20 mM Tris-HC1, pH 7. The antibody elutes around 500-400 mM (NH₄)₂SO₄. The pooled protein was dialyzed against PBS and stored a −80° C.

N-glycan analysis—N-glycan analysis was carried out by liquid-chromatography electrospray ionization-mass spectrometry (LC-ESI-MS) of tryptic glycopeptides [37]. In short, bands that correspond to the heavy chain in a Coomassie stained SDS-PAGEs were excised, proteins S alkylated, digested with trypsin and subsequently analysed by LC-ESI-MS [37].

Biacore analyses—Recombinant human FcγRI and FcγRIII (Sino Biological, China) were immobilized onto the surface of CM5 chips (GE Healthcare) using an amine-coupling kit with a target capture level of 1000 RUs. Each mAb (diluted in HBS-EP+buffer; GE Healthcare) was then flowed over the chip at 5 different concentrations (with the highest concentration having an Rmax between 30-80 RUs) and kinetic analyses using BIAEvaluation software performed (1:1 fit). Fast flow rates and controls (including a flow cell with no receptor, and immobilized receptor with flow of buffer only) were performed to insure against acquiring mass transfer-limited data. Binding data with HIS-tagged murine Fcγ receptors (Sino Biological, China) were generated using a NTA sensor chip. Briefly, approximately 1000 RUs of receptor was captured on the chip followed by a flow of h-13F6 mAb at a fixed concentration (5 μg/m1). For determining C1q affinity, a protein A (Pierce Biotechnology) CM5 biosensor chip (GE Healthcare) was generated using a standard primary amine coupling protocol. The chip's reference channel was coupled to bovine serum albumin (BSA) to minimize nonspecific binding of C1q. Antibodies at 50 nM were immobilized on the protein A surface for 0.5 or 1 min at 10 gL/min. C1q in 2-fold serial dilutions (starting at 100 or 25 nM, 5 concentrations total) was injected over antibody-bound surface for 3 min at 30 gL/min followed by a 4.5 min dissociation phase. C1q molarity was calculated using the molecular weight of the C1q hexameric bundle, 410 kDa.

C1q binding ELISA—The binding of human C1q (Calbiochem; San Diego, Calif.) to IgG mAb was assessed by a method previously described [38]. High binding Costar 96-well plates (Cambridge, Mass.) were coated overnight at 4° C. with various concentrations of mAb diluted in coating buffer (PBS). After blocking (PBS/2% BSA) for 1 hour, 2 μg/ml of human C1q was added. The binding of C1q to the mAb was detected using a 1/1000 dilution of goat anti-human C1q polyclonal antibody (Calbiochem) followed with a 1/5000 dilution of rabbit anti-goat (human adsorbed) HRP conjugated antibody (Southern Biotech; Birmingham, Al.). The plates were developed with TMB (KPL; Gaithersburg, Md.). The reaction was stopped with 2.5 N H₂50₄, and the absorbance at 450 nm was measured.

Virus, animals, and infections—Mouse-adapted EBOV virus was obtained from Dr. Mike Bray [39]. Female C57BL/6 or BALB/c mice (5-8 weeks old) were obtained from the National Cancer Institute (Frederick, Md.) and housed under specific pathogen-free conditions. For infection, mice were inoculated i.p. with 1000 PFU (30,000 LD₅₀) of mouse-adapted EBOV virus in a biosafety level 4 (BSL-4) laboratory. Animals were observed at least daily for 28 days following exposure to the virus. Research was conducted in compliance with the Animal Welfare Act and other federal statutes and regulations relating to animals and experiments involving animals, and adheres to principles stated in the Guide for the Care and Use of Laboratory Animals, National Research Council, 1996. The facility where this research was conducted is fully accredited by the Association for the Assessment and Accreditation of Laboratory Animal Care International.

Statistics—Survival curves are analyzed with the Log-Rank (Mantel-Cox) test. Affinities are compared with an unpaired T-test (2-sided). Logistic regression models are used to obtain the point estimate and confidence interval for ED₅₀ where the dependent variable was the logit of the probability of survival, and the independent variable was the log of dose. ED₅₀ was estimated by using negative of the ratio of the model's intercept to the slope (the regression estimate of the log dose). The standard error of the estimated ED₅₀ is calculated using the model's variance-covariance matrix of the estimated intercept and slope. Relative potency, a quantity defined as the ratio of two ED₅₀ and its 95% confidence interval are estimated using logistic regression models with two intercepts, and a common slope (for the two compared assays), under the assumption of parallel lines. The estimate of the relative potency is the ratio of the difference of the two intercepts to the slope estimate. Fieller's theorem [40] are used to derive the 95% confidence interval for the relative potency estimate. All analyses are performed using Prism software (GraphPad) and SAS software [41].

### Example I

### Novel Glycoforms on mAbs Produced in CHO, Nicotiana and Yeast

Three mAbs were used in order evaluate the effects of different glycoforms on FcγR and c1q binding. The mAbs and their N-linked glycans are listed in Table 2. Their specificities respresent an anti-viral mAb (mAb 13F6, anti-Ebola virus [42], an anti-B cell mAb (anti-CD20, rituximab [43]) and an anti-tumor mAb (anti-HER2, trastuzumab [44]). The various mAb glycoforms were produced in three different systems. The first system, Chinese hamster ovarian (CHO) cells, are currently the most commonly used platform to manufacture FDA approved recombinant mAbs. A stable wild-type CHO line was used to produce the mAbs that contained typical glycans (+fucose and galactose) commonly found on recombinant antibodies. A second CHO line (lec8 [48]) was used to produce mAbs that were devoid of galactose residues. In order to inhibit fucose glycosylation in this line, a gene knockout approach was used as previously described [36] resulting in a CHO-lec8 cell line where the expressed mAbs were predominantly the GNGN glycoform, with no fucose or galactose residues. A third CHO line (lec13 [49]) was used to produce mAbs that were predominantly of the G1/G2 glycoform with no fucose residues.

The second production method is a transient plant system (magnICON®) in which the heavy and light chain genes are cloned into separate vectors containing different but compatible viral replicons to allow for the simultaneous expression of heavy and light chains from replicating viral segments. The heavy and light chain vectors are then introduced into Agrobacterium tumefaciens to allow for high efficiency infection of one month old Nicotiana benthamiana plants by vacuum infiltration. In order to generate mAbs with novel glycoforms, the Nicotiana benthamiana plants used for Agrobacterium infection and subsequent antibody production is in turn modified by the TALE gene knockout technique to eliminate the expression of the endogenous plant-specific xylosyl and fucosyl transferase genes [36]. An additional glycoform is created by co-infection of plants with Agrobacterium containing a galactosyl transferase gene functional in plant cells. The resulting three mAb glycoforms are wild-type, minus fucose and galactose (-FG), and minus fucose (-F).

The third production method utilized the yeast Pichia pastoris for the assembly and glycosylation of the mAbs. Two glycoengineered yeast lines were prepared using well-known methods previously described [11]. The first glycoengineered line (delta-ochl, delta-pno1, delta-mnn4B, delta-bmt2, Kluyveromyces lactis UDP-G1cNAc transporter, alpha-1,2 Mus musculus MnsI, beta-1,2 G1cNAc transferase I, beta-1,2 Rattus norvegicus G1cNAc transferase II, Drosophila melanogaster MnsII, Schizosaccharomyces pombe Gal epimerase, D. melanogaster UDP-Gal transporter, Homo sapiens beta-1,4 galactosyl transferase, alpha-1,3 fucosyltransferase, Arabidopsis thaliana GDP-4-keto-6-deoxymannose-3,5-epimerase-4-reductase and Arabidopsis thaliana GDP-mannose-4,6-dehydratase) was used to produce mAb glycoforms containing fucose and galactose added to the core glycan, and referred to as +GF yeast. The second glycoengineered line contained all of the same genes except for those involved in fucosylation and galactosylation (Schizosaccharomyces pombe Gal epimerase, D. melanogaster UDP-Gal transporter, Homo sapiens beta-1,4 galactosyl transferase, alpha-1,3 fucosyltransferase, Arabidopsis thaliana GDP-4-keto-6-deoxymannose-3,5-epimerase-4-reductase and Arabidopsis thaliana GDP-mannose-4,6-dehydratase) resulting in mAb glycoforms containing predominantly GNGN glycan referred to as -GF yeast.

In addition to the mAb DNA used for expression in CHO cells, plants, or yeast, we made an additional construct containing an alanine at asparagine 297 of the human 13F6 IgG₁ heavy chain constant region (N297A mutation) to eliminate Fc glycosylation entirely. The aglycosylated mAb was produced in the plant system.

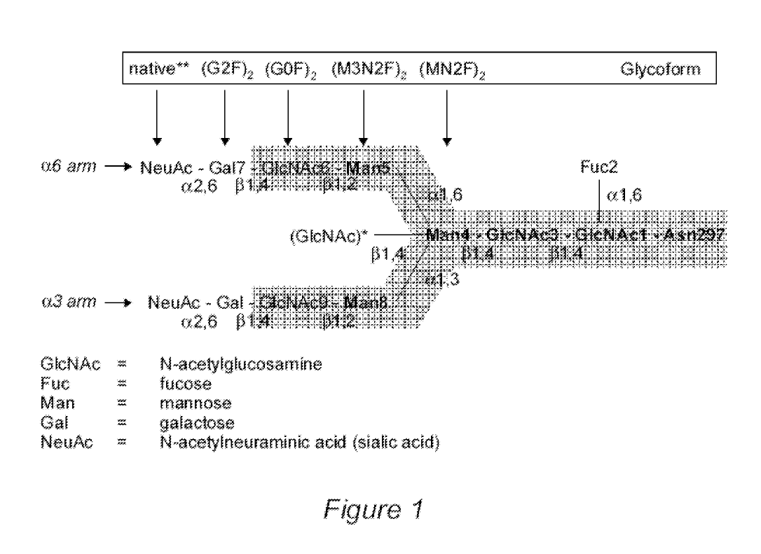

A representation of a core glycan is shown in FIG. 1 and the structures of produced glycans is provided in Table 1. The distribution of N-linked glycans on the mAbs produced by the various production methods is shown in Table 2.

To determine the glycoforms of these mAb variants, mAbs were purified by Protein A affinity chromatography and subjected to N-glycosylation analysis using LC-ESI-MS [35]. The N-glycosylation profile of the CHO-derived mAbs exhibited 3-4 glycoforms. The N-glycosylation profile of the plant-derived mAbs exhibited 3-9 glycoforms. The N-glycosylation profile of the yeast-derived mAbs exhibited 2-7 glycoforms. In all cases where fucosyl transferase was absent, the predominant glycoform was GNGN or G1/G2. In the presence of fucosyl transferase, the G0, and G0X glycoforms predominated. In contrast, no fucosylated structures were detected in mAbs produced in any system without fucosyl transferase. As expected, no glycan structures were detected in the aglycosylated mAbs.

TABLE 1
Structures of glycans

| Glycan Name | N297-Glycan Structures |
|---|---|
| AGLY | Aglycosylated mAb |
| MAN 5 | GlcNac₂Man₅ |
| MAN 6-12 | GlcNac₂Man₆₋₁₂ |
| GN | GlcNac₂Man₃GlcNac |
| GNF | GlcNac₂Man₃GlcNac + fucose |
| GNGN | GlcNac₂Man₃GlcNac₂ |
| G0 | GlcNac₂Man₃GlcNac₂ + fucose |
| G1 | GlcNac₂Man₃GlcNac₂Gal |
| G1F | GlcNac₂Man₃GlcNac₂Gal + fucose |
| G2 | GlcNac₂Man₃GlcNac₂Gal₂ |
| G2F | GlcNac₂Man₃GlcNac₂Gal₂ + fucose |
| G0X | GlcNac₂Man₃GlcNac₂ + fucose + xylose |
| GNGNX | GlcNac₂Man₃GlcNac₂ + xylose |

GlcNac = N-acetylglucosamine
Man = Mannose
Gal = Galactose
Core glycan = GlcNac₂Man₃GlcNac₂
+ Fucose = fucose attached to GlcNac #1 in core
+ Bisecting Man = a third Man attached to core Man #1
+ Xylose = Xylose attached to core Man #1

TABLE 2
Distribution of N-linked glycans on mAbs (mol %)

| MAb | AGLY | MAN 5 | MAN 6-12 | GN | GNF | GNGN | G0 | G1 | G1F | G2 | G2F | G0X | GNGNX |
|---|---|---|---|---|---|---|---|---|---|---|---|---|---|
| 1 | 5 | | | 5 | | 90 | | | | | | | |
| 2 | | | | | | | | 5 | | 95 | | | |
| 3 | 4 | 14 | 10 | 6 | 8 | 6 | 12 | | | | | 35 | 5 |
| 4 | 4 | 3 | | | | 93 | | | | | | | |
| 5 | | | | | | | | 13 | | 87 | | | |
| 6 | 3 | 12 | 11 | 7 | 12 | 5 | 10 | | | | | 36 | 4 |
| 7 | 3 | | | 8 | | 85 | | | | | | | |
| 8 | | | | | | | | 34 | | 46 | | | |
| 9 | | | | | 4 | | 53 | | 35 | | 8 | | |
| 10 | | 6 | | 3 | | 91 | | | | | | | |
| 11 | | | | | | | | 63 | | 37 | | | |
| 12 | | 6 | | | 4 | 8 | 60 | 8 | 7 | | 7 | | |
| 13 | 100 | | | | | | | | | | | | |

MAb numbers and sources:
1 Anti-Ebola virus produced in −XF plants
2 Anti-Ebola virus produced in −XF +gal plants
3 Anti-Ebola virus produced in wild type plants
4 Anti-CD20 produced in −XF plants
5 Anti-CD20 produced in −XF + gal plants
6 Anti-CD20 produced in wild type plants
7 Anti-CD20 produced in −F −gal CHO cells
8 Anti-CD20 produced in −F + gal CHO cells
9 Anti-CD20 produced in wild type CHO cells
10 Anti-HER2 produced in −gal and −fuc yeast
11 Anti-HER2 produced in − fuc +gal yeast
12 Anti-HER2 produced in +gal +fuc yeast
13 Anti-Ebola virus (N297A) produced in −XF plants

### Example II

### Affinities of mAbs for Fc Receptors and C1q

Affinity of mAbs for FcγRI (CD64)—Surface plasmon resonance (SPR) was performed to determine the affinities of mAbs for recombinant human FcγRI (Table 3), a receptor important for ADCC [3,10]. In general, mAbs lacking fucose have significantly higher affinity for human FcγRI compared to fucosylated mAbs (P<0.05 in all cases). Binding by the aglycosylated mAb was significantly (P<0.05) lower than all the other mAbs tested.

TABLE 3
KD (×10⁻⁸M)

| MAb | FcγRI (CD64) | FcγRIIIA (CD16) |
|---|---|---|
| 1 | 1.6 ± 0.3 | 2.5 ± 0.3 |
| 2 | 1.5 ± 0.3 | 2.6 ± 0.3 |
| 3 | 4.2 ± 1.2 | 7.2 ± 1.2 |
| 4 | 1.5 ± 0.4 | 2.4 ± 0.3 |
| 5 | 1.6 ± 0.3 | 2.7 ± 0.3 |
| 6 | 4.3 ± 1.2 | 7.3 ± 1.2 |
| 7 | 1.4 ± 0.4 | 2.6 ± 0.3 |
| 8 | 1.3 ± 0.4 | 2.5 ± 0.3 |
| 9 | 4.2 ± 0.7 | 15 ± 1.6 |
| 10 | 1.4 ± 0.4 | 2.3 ± 0.3 |
| 11 | 1.5 ± 0.4 | 2.4 ± 0.3 |
| 12 | 4.5 ± 1.3 | 7.5 ± 1.4 |
| 13 | 34 ± 16 | 12 ± 1.1 |

Affinity of mAbs for human FcγRIIIA (CD16)—Surface plasmon resonance was also performed with recombinant FcγRIIIA (Table 3), a receptor important for induction of ADCC by NK cells [47]. Among all mAbs, the aglycosylated mAb had the weakest affinity (12±1.1×10⁻⁸ M). As with FcγRI, the afucosylated mAbs had significantly higher affinities (2-3×10⁻⁸ M) compared to fucosylated mAbs (7-15×10⁻⁸ M). Notably, the values for the afucosylated mAbs are all high affinities for what is traditionally considered a low to medium affinity receptor.

C1q binding by mAbs—C1q binding to the Fc region of antibodies, the first step in the classical complement cascade, is glycosylation dependent. The ability of the different mAbs to bind human C1q was compared (Table 4) using a standard ELISA assay at a constant 2.5 μg/ml of antibody [38] as well as surface plasmon resonance (SPR) to compare C1q affinities. As expected, the aglycosylated mAb did not bind C1q at the concentration tested (2.5 μg/ml). In contrast, binding of both fucosylated and afucosylated mAbs was observed, and the afucosylated mAbs were significantly less potent binders compared to fucosylated mAbs at that mAb concentration.

TABLE 4
C1q Binding ELISA

| MAb | Absorbance |
|---|---|
| 1 | 0.49 ± 0.06 |
| 2 | 0.96 ± 0.10 |
| 3 | 0.92 + +0.09 |
| 4 | 0.48 ± 0.07 |
| 5 | 1.00 ± 0.11 |
| 6 | 0.94 + 0.10 |
| 7 | 0.51 ± 0.07 |
| 8 | 1.10 ± 0.12 |
| 9 | 0.75 + 0.08 |
| 10 | 0.53 + 0.05 |
| 11 | 1.05 + 0.09 |
| 12 | 0.96 + 0.12 |
| 13 | 0.01 + 0.01 |

MAbs at 2.5 μg/ml

SPR analysis of C1q binding to antibodies revealed that the mAbs produced to be glycoforms containing galactose (#s 2,3,5,6,8,9,11,12) all had Kds in excess of 100 nM (>100×10⁻⁹ M) whereas mAb glycoforms that were devoid of fucose and galactose (#s 1,4,7,10) had Kd values of approximately 50 nM (50×10⁻⁹ M).

### Example III

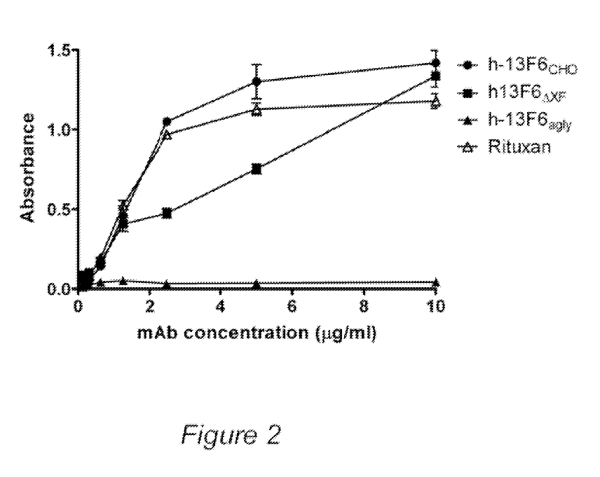

Efficacy of mAbs against lethal Ebola challenge—To determine whether the different N-glycoforms present on the Fc region of these mAbs have an effect on efficacy in vivo, the plant-derived variants of an anti-Ebola mAb (mAbs #1 and #2) were tested in a well-established lethal EBOV challenge model. The anti-Ebola mAb used in the survival study is referred to as h-13F6. Groups of mice (n=10) received single intraperitoneal doses of mAb followed by a lethal challenge (1,000 pfu˜30,000 LD₅₀). The resulting dose response data are shown in FIG. 2. Although a highly lethal challenge was administered, 20% of control mice survived. This is a common observation since the institution of the IACUC requirement that mice displaying significant morbidity be treated with a DietGel nutritional supplement. h-13F6_{ΔXF} was more protective (ED₅₀=3 μg˜0.15 mg/kg) than h-13F6_{WT} (ED₅₀=11 μg˜0.55 mg/kg). As would be expected if the protective activity of the mAb involves any Fc-mediated effector function mechanisms, the aglycosylated h-13F6_{agly} (mAb #13) provided less protection (ED₅₀=33 μg˜1.65 mg/kg). Relative potency was significantly different between h-13F6_{ΔXF} and h-13F6_{wT} (relative potency: 0.26, 95%CI: 0.07-0.91) and between h-13F6_{ΔXF} and h-13F6_{agly} (relative potency: 10.72, 95%CI: 2.10-81.53).

Survival curves for the low dose groups (3 μg˜0.2 mg/kg) demonstrated that mice receiving h-13F6_{ΔXF} were significantly protected (median survival of 18.5 days) when compared with h-13F6_{wT} (P<0.05; median survival of 7 days) and the negative PBS control (P<0.05; median survival of 6 days).

### Equivalents

While this invention has been particularly shown and described with references to preferred embodiments thereof, it will be understood by those skilled in the art that various changes in form and details may be made therein without departing from the scope of the invention encompassed by the appended claims.

The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein.

In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. The disclosures of all articles and references, including patent publications, are incorporated herein by reference.

### REFERENCES

The following references are cited in the specification by reference number; all of these references are incorporated by this reference in the application in their entirety:
1. Reichert J M. Antibody-based therapeutics to watch in 2011. MAbs. 2011 Jan-Feb;3(1):76-99.
2. Beck A, Haeuw J F, Wurch T, Goetsch L, Bailly C, CorvaIa N. The next generation of antibody-drug conjugates comes of age. Discov Med. 2010 Oct;10(53):329-39.
3. Jefferis R. Antibody therapeutics: isotype and glycoform selection. Expert Opin Biol Ther. 2007;7(9):1401-13.
4. Shields, R. L., et al. Lack of fucose on human IgG1 N-linked oligosaccharide improves binding to human FcγRIII and antibody-dependent cellular toxicity, J. Biol. Chem., Vol. 277, Issue 30, 26733-26740, Jul. 26, 2002.
5. Shinkawa, T. et al. The absence of fucose but not the presence of galactose or bisecting N-acetylglucosamine of human IgG1 complex-type oligosaccharides shows the critical role of enhancing antibody-dependent cellular cytotoxicity. J. Biol. Chem., January 2003; 278: 3466-3473.
6. Niwa, R., et al. Defucosylated chimeric Anti-CC chemokine receptor 4 IgG1 with

Enhanced Antibody-Dependent Cellular Cytotoxicity Shows Potent therapeutic activity to T-Cell leukemia and lymphoma. Cancer Res. 2004 64: 2127-2133.
7. Wu, J., et al., A novel polymorphism of FcgRIIIa (CD16a) alters receptor function and predisposes to autoimmune disease. J Clin Invest, 1997. 100(5): p. 1059-70.
8. Weng, W. K. and R. Levy, Two immunoglobulin G fragment C receptor polymorphisms independently predict response to rituximab in patients with follicular lymphoma. J Clin Oncol, 2003. 21(21): p. 3940-7.
9. Weng, W. K., et al., Clinical outcome of lymphoma patients after idiotype vaccination is correlated with humoral immune response and immunoglobulin G Fc receptor genotype. J Clin Oncol, 2004.22(23): p. 4717-24.
10. Jefferis, R. 2005. Glycosylation of recombinant antibody therapeutics. Biotechnol Prog 21:11-16.
11. Li, H., Sethuraman, N., Stadheim, T. A, Zha, D., Prinz, B., Ballew, N., Bobrowicz, P., Choi, B. K., Cook, W. J., Cukan, M., Houston-Cummings, N. R., Davidson, R., Gong, B., Hamilton, S. R., Hoopes, J. P., Jiang, Y., Kim, N., Mansfield, R., Nett, J. H., Rios, S., Strawbridge, R., Wildt, S., and Gerngross, T. U. 2006. Optimization of humanized IgGs in glycoengineered Pichia pastoris. Nat Biotechnol 24:210-215.
12. Lund, J., Takahashi, N., Popplewell, A., Goodall, M., Pound, J. D., Tyler, R., King, D. J., and Jefferis, R. 2000. Expression and characterization of truncated forms of humanized L243 IgGl. Architectural features can influence synthesis of its oligosaccharide chains and affect superoxide production triggered through human Fc-g receptor I. Eur J Biochem 267:7246-7257.
13. Mimura, Y., Church, S., Ghirlando, R., Ashton, P. R., Dong, S., Goodall, M., Lund, J., and Jefferis, R. 2000. The influence of glycosylation on the thermal stability and effector function expression of human IgG1 -Fc: properties of a series of truncated glycoforms. Mol Immunol 37:697-706.
14. Krapp, S, Mimura, Y, Jefferis, R., Huber, R, and Sondermann, P. 2003. Structural analysis of human IgG-Fc glycoforms reveals a correlation between glycosylation and structural integrity. J Mol Biol 325:979-989.
15. Mimura, Y, Sondermann, P., Ghirlando, R., Lund, J, Young, S. P., Goodall, M, and

Jefferis, R. 2001. Role of oligosaccharide residues of IgG1-Fc in FcγRllb binding. J Biol Chem 276:45539-45547.
16. Sondermann, P., Huber, R., Oosthuizen, V., and Jacob, U. 2000. The 3.2-A crystal structure of the human IgG1 Fc fragment-Fc gRIII complex. Nature 406:267-273.
17. Lifely, M. R., Hale, C, Boyce, S., Keen, M. J., and Phillips, J. 1995. Glycosylation and biological activity of CAMPATH-1H expressed in different cell lines and grown under different culture conditions. Glycobiology 5:813-822.
18. Umana, P., Jean-Mairet, J., Moudry, R., Amstutz, H., and Bailey, J. E. 1999. Engineered glycoforms of an antineuroblastoma IgG1 with optimized antibody-dependent cellular cytotoxic activity. Nat Biotechnol 17:176-180.
19. Davies, J., Jiang, L., Pan, L. Z., LaBarre, M. J., Anderson, D., and Reff, M. 2001. Expression of GnTIII in a recombinant anti-CD20 CHO production cell line: Expression of antibodies with altered glycoforms leads to an increase in ADCC through higher affinity for FCyRIII. Biotechnol Bioeng 74:288-294.
20. Shinkawa, T., Nakamura, K., Yamane, N., Shoji-Hosaka, E., Kanda, Y, Sakurada, M., Uchida, K., Anazawa, H., Satoh, M., Yamasaki, M., Hanai, N., and shiara, K. 2003. The absence of fucose but not the presence of galactose or bisecting N-acetylglucosamine of human IgG1 complex-type oligosaccharides shows the critical role of enhancing antibody-dependent cellular cytotoxicity. J Biol Chem 278:3466-3473.
21. Rumpel, B. M., Rademacher, T. W., Rook, G. A., Williams, P. J., and Wilson, I. B. 1994. Galactosylation of human IgG monoclonal anti-D produced by EBV-transformed B-lymphoblastoid cell lines is dependent on culture method and affects Fc receptor-mediated functional activity. Hum Antibodies Hybridomas 5:143-151.
22. Rumpel, B. M, Wang, Y, Griffiths, H. L, Hadley, A. G., and Rook, G. A. 1995. The biological activity of human monoclonal IgG anti-D is reduced by beta-galactosidase treatment. Hum Antibodies Hybridomas 6:82-88.
23. Boyd, P. N, Lines, A. C, andPatel, A. R. 1995. The effect of the removal of sialic acid, galactose and total carbohydrate on the functional activity of Campath-IH. Mol Immunol 32:1311-1318.
24. Lund, J., Takahashi, N., Pound, J. D., Goodall, M., Nakagawa, H., and Jefferis, R. 1995. Oligosaccharide protein interactions in IgG can modulate recognition by Fcg receptors. Faseb J 9:115-119.
25. Stanley, P. 1992. Glycosylation engineering. Glycobiology 2:99-107.
26. Hollister, J., Grabenhorst, E., Nimtz, M., Conradt, H., and Jarvis, D. L. 2002. Engineering the protein N-glycosylation pathway in insect cells for production of biantennary, complex N-glycans. Biochemistry 41:15093-15104.
27. Choi, B. R., Bobrowicz, P., Davidson, R. C, Hamilton, S. R, Rung, D. H, Li, H, Miele, R. G, Nett, J. H., Wildt, S., and Gerngross, T. U. 2003. Use of combinatorial genetic libraries to humanize N-linked glycosylation in the yeast Pichia pastoris. Proc Natl Acad Sci USA 100: 5022-5027.
28. Hamilton, S. R., Bobrowicz, P., Bobrowicz, B., Davidson, R. C, Li, H., Mitchell, T., Nett, J. H., Rausch, S., Stadheim, T. A., Wischnewski, H., Wildt, S., and Gerngross, T. U. 2003. Production of complex human glycoproteins in yeast. Science 301:1244-1246.
29. Wildt, S, and Gerngross, T. U. 2005. The humanization of N-glycosylation pathways in yeast. Nat Rev Microbiol 3:119-128.
30. Niwa, R., Sakurada, M., Robayashi, Y, Uehara, A., Matsushima, R., Ueda, R., Nakamura, R., and shiara, R. 2005. Enhanced natural killer cell binding and activation by low-fucose IgG1 antibody results in potent antibody-dependent cellular cytotoxicity induction at lower antigen density. Clin Cancer Res 11:2327-2336.
31. Yamane-Ohnuki, N., Rinoshita, S., Inoue-Urakubo, M., Rusunoki, M., Iida, S., Nakano, R., Wakitani, M., Niwa, R., Sakurada, M., Uchida, R., shiara, R., and Satoh, M. 2004. Establishment of FUT8 knockout Chinese hamster ovary cells: an ideal host cell line for producing completely defucosylated antibodies with enhanced antibody-dependent cellular cytotoxicity. Biotechnol Bioeng 87:614-622.
32. Gerngross T. Production of complex human glycoproteins in yeast. Adv Exp Med Biol. 2005;564.
33. Strasser R, Stadlmann J, Schahs M, Stiegler G, Quendler H, Mach L, Glossl J, Weterings K, Pabst M, Steinkellner H (2008) Generation of glycol-engineered Nicotiana benthamiana for the production of monoclonal antibodies with a homogeneous human-like N-glycan structure. Plant Biotech J 6:392-402.
34. Giritch A, Marillonnet S, Engler C, van Eldik G, Botterman J, Klimyuk V, Gleba Y. Rapid high-yield expression of full-size IgG antibodies in plants coinfected with noncompeting viral vectors. Proc Natl Acad Sci USA. 2006;103(40):14701-6.
35. Hiatt, A. and Pauly, M. Monoclonal antibodies from plants: A new speed record, PNAS 2006 103:14645-14646.
36. Li, T. et al. Modularly assembled designer TAL effector nucleases for targeted gene knockout and gene replacement in eukaryotes. Nucleic Acids Research, 2011, Vol. 39, No. 14 6315-6325
37. Stadlmann J, Pabst M, Kolarich D, Kunert R, Altmann F (2008) Analysis of immunoglobulin glycosylation by LC-ESI-MS of glycopeptides and oligosaccharides. Proteomics 8:2858-71.
38. Idusogie E E, et al. (2001) Engineered antibodies with increased activity to recruit complement. J Immunol 166:2571-2575.
39. Bray M, Davis K, Geisbert T, Schmaljohn C, Huggins J (1998) A mouse model for evaluation of prophylaxis and therapy of Ebola hemorrhagic fever. J Infect Dis 178:651.
40. Zerbe G O (1978) On Fieller's theorem and the general linear model. Am Stat 32.
41. SAS Institute Inc (2003) SAS/STAT Software, Version 8, Cary N.C.
42. Zeitlin, L. et al. 2011. Enhanced potency of a fucose-free monoclonal antibody being developed as an Ebola virus immunoprotectant. Proc. Natl. Acad. Sci. USA, in press.
43. Main C, Pitt M, Moxham T, Stein K. The clinical effectiveness and cost-effectiveness of rituximab for the first-line treatment of chronic lymphocytic leukaemia: an evidence review of the submission from Roche. Health Technol Assess. 2010 Oct;14(Suppl. 2):27-32.
44. Chang HR. Trastuzumab-based neoadjuvant therapy in patients with HER2-positive breast cancer. Cancer. 2010 Jun 15;116(12):2856-67.
45. Jochen G Salfeld. (2007) Isotype selection in antibody engineering. Nat Biotechnol 25 (12), 1369-1372.
46. Forthal DN, et al. (2010) Fc-glycosylation influences Fcγ receptor binding and cell-mediated anti-HIV activity of monoclonal antibody 2G12. J Immunol 185:6876-6882.
47. Moore, G. L., Chen, H., Karki S. and Lazar, G. A. Engineered Fc variant antibodies with enhanced ability to recruit complement and mediate effector functions. mAbs 2:2, 181-189; March/April, 2010.
48. Wright A, Morrison SL. Effect of C2-associated carbohydrate structure on Ig effector function: studies with chimeric mouse-human IgG1 antibodies in glycosylation mutants of Chinese hamster ovary cells. J Immunol. 1998 Apr. 1;160(7):3393-402.
49. Wang L W, Dlugosz M, Somerville R P, Raed M, Haltiwanger R S, Apte S S. 0-fucosylation of thrombospondin type 1 repeats in ADAMTS-like-l/punctin-1 regulates secretion: implications for the ADAMTS superfamily. J Biol Chem. 2007 Jun. 8;282(23):17024-31.
50. Nimmerjahn F, Ravetch J (2006). “Fcgamma receptors: old friends and new family members”. Immunity 24 (1): 19-28.
51. Perussia B, Dayton E, Lazarus R, Fanning V, Trinchieri G (1983). “Immune interferon induces the receptor for monomeric IgG1 on human monocytic and myeloid cells”. J Exp Med 158 (4): 1092-113.
52. Repp R, Valerius T, Sendler A, Gramatzki M, Iro H, Kalden J, Platzer E (1991). “Neutrophils express the high affinity receptor for IgG (Fc gamma RI, CD64) after in vivo application of recombinant human granulocyte colony-stimulating factor”. Blood 78 (4): 885-9.
53. Swanson J, Hoppe A (2004). “The coordination of signaling during Fc receptor-mediated phagocytosis”. J Leukoc Biol 76 (6): 1093-103.
54. Joshi T, Butchar J, Tridandapani S (2006). “Fcgamma receptor signaling in phagocytes”. Int J Hematol 84 (3): 210-6.
55. Janeway, Charles (2001). “Appendix II. CD antigens”. Immunobiology (5 ed.). New York: Garland.
56. Trinchieri G, Valiante N (1993). “Receptors for the Fc fragment of IgG on natural killer cells”. Nat Immun 12 (4-5): 218-34.
57. Sulica A, Galatiuc C, Manciulea M, Bancu A, DeLeo A, Whiteside T, Herberman R (1993). “Regulation of human natural cytotoxicity by IgG. IV. Association between binding of monomeric IgG to the Fc receptors on large granular lymphocytes and inhibition of natural killer (NK) cell activity”. Cell Immunol 147 (2): 397-410.
58. Tschopp, J., Villiger, W., Lustig, A., Jaton, J. C., and Engel, J. (1980) Eur. J. Immunol. 10,529-535.
59. Poon, P. H., Phillips, M. L., and Schumaker, V. N. (1985) J. Biol. Chem. 260, 9357-9365.
60. Hughes-Jones, N. C., and Gardner, B. (1979) Mol. Immunol. 16, 697-701.
61. Painter, R. H. (1993) Behring Inst. Mitt. 93, 131-137.
62. Loos, M. (1983) Curr. Top. Microbiol. Immunol. 102, 1-56.
63. Bindon, C. I., Hale, G., Bruggemann, M., and Waldmann, H. (1988) J. Exp. Med. 168, 127-142.

We claim:

1. An antibody, or antigen binding fragment thereof, wherein the antibody is present in a substantially homogeneous composition represented by the presence of the GNGN glycoform and wherein the antibody has a binding affinity for human FcγRI and FcγRIIIa and said binding affinities for FcγRI and FcγRIIIa of the GNGN antibody, are greater than the binding affinities for FcγRI and FcγRIIIa of the antibody, or antigen binding fragment thereof present in a composition containing G0, G1F, G2F or GNGNF glycoforms.

2. An antibody, or antigen binding fragment thereof, wherein the antibody is present in a substantially homogenous composition represented by the presence of the GNGN glycoform and
a. wherein the antibody, or antigen binding fragment thereof, has a binding affinity for human C1q and FcγRIIIa and
b. wherein said binding affinities for C1q of the GNGN antibody, or antigen binding fragment thereof, is less than the binding affinities for C1q of the antibody, or antigen binding fragment thereof present in a composition containing G0, G1F, G2F or GNGNF glycoforms and
c. wherein said binding affinity for FcγRIIIa of the GNGN antibody, or antigen binding fragment thereof, is greater than the binding affinity for FcγRIIIa of the antibody, or antigen binding fragment thereof present in a composition containing G0, G1F, G2F or GNGNF glycoforms.

3. An antibody, or antigen binding fragment thereof, wherein the antibody is present in a substantially homogenous composition represented by the presence of the GNGN glycoform and
a. wherein the antibody, or antigen binding fragment thereof, has a binding affinity for C1q and FcγRI and
b. wherein said binding affinities for C1q of the GNGN antibody, or antigen binding fragment thereof, is less than the binding affinities for C1q of the antibody, or antigen binding fragment thereof present in a composition containing G0, G1F, G2F or GNGNF glycoforms and
c. wherein said binding affinity for FcγRI of the GNGN antibody, or antigen binding fragment thereof, is greater than the binding affinity for FcγRI of the antibody, or antigen binding fragment thereof present in a composition containing G0, G1F, G2F or GNGNF glycoforms.

4. An antibody, or antigen binding fragment thereof, wherein the antibody is present in a substantially homogenous composition represented by the presence of the GNGN glycoform and
a. wherein the antibody, or antigen binding fragment thereof, has a binding affinity for human FcγRI, FcγRIIIa and C1q, and
b. wherein said binding affinities for FcγRI and FcγRIIIa of the GNGN antibody, or antigen binding fragment thereof, are greater than the binding affinities for FcγRI and FcγRIIIa of the antibody, or antigen binding fragment thereof present in a composition containing G0, G1F, G2F or GNGNF glycoforms and
c. wherein said binding affinity for C1q of the GNGN antibody, or antigen binding fragment thereof, is less than the binding affinity for C1q of the antibody, or antigen binding fragment thereof present in a composition containing G0, G1F, G2F or GNGNF glycoforms.

5. An antibody, or antigen binding fragment thereof, wherein the antibody is present in a substantially homogeneous composition represented by the presence of the G1/G2 glycoform and wherein the antibody has a binding affinity for human FcγRI and FcγRIIIa and said binding affinities for FcγRI and FcγRIIIa of the G1G2 antibody, are greater than the binding affinities for FcγRI and FcγRIIIa of the antibody, or antigen binding fragment thereof present in a composition containing G0, G1F, G2F or GNGNF glycoforms.

6. An antibody, or antigen binding fragment thereof, wherein the antibody is present in a substantially homogenous composition represented by the presence of the G1/G2 glycoform and
a. wherein the antibody, or antigen binding fragment thereof, has a binding affinity for human C1q and FcγRIIIa and
b. wherein said binding affinities for C1q of the G1/G2 antibody, or antigen binding fragment thereof, is less than the binding affinities for C1q of the antibody, or antigen binding fragment thereof present in a composition containing G0, G1F, G2F or GNGNF glycoforms and
c. wherein said binding affinity for FcγRIIIa of the G1/G2 antibody, or antigen binding fragment thereof, is greater than the binding affinity for FcγRIIIa of the antibody, or antigen binding fragment thereof present in a composition containing G0, G1F, G2F or GNGNF glycoforms.

7. An antibody, or antigen binding fragment thereof, wherein the antibody is present in a substantially homogenous composition represented by the presence of the G1/G2 glycoform and
a. wherein the antibody, or antigen binding fragment thereof, has a binding affinity for C1q and FcγRI and
b. wherein said binding affinities for C1q of the G1/G2 antibody, or antigen binding fragment thereof, is less than the binding affinities for C1q of the antibody, or antigen binding fragment thereof present in a composition containing G0, G1F, G2F or GNGNF glycoforms and
c. wherein said binding affinity for FcγRI of the G1/G2 antibody, or antigen binding fragment thereof, is greater than the binding affinity for FcγRI of the antibody, or antigen binding fragment thereof present in a composition containing G0, G1F, G2F or GNGNF glycoforms.

8. An antibody, or antigen binding fragment thereof, wherein the antibody is present in a substantially homogenous composition represented by the presence of the G1/G2 glycoform and
a. wherein the antibody, or antigen binding fragment thereof, has a binding affinity for human FcγRI, FcγRIIIa and C1q, and
b. wherein said binding affinities for FcγRI and FcγRIIIa of the G1/G2 antibody, or antigen binding fragment thereof, are greater than the binding affinities for FcγRI and FcγRIIIa of the antibody, or antigen binding fragment thereof present in a composition containing G0, G1F, G2F or GNGNF glycoforms and
c. wherein said binding affinity for C1q of the G1/G2 antibody, or antigen binding fragment thereof, is less than the binding affinity for C1q of the antibody, or antigen binding fragment thereof present in a composition containing G0, G1F, G2F or GNGNF glycoforms.

9. The antibody, or antigen binding fragment thereof, of claims 1-8 wherein said antibody is produced in a plant.

10. The antibody, or antigen binding fragment thereof, of claims 1-8 wherein said antibody binds to a virus.

11. The antibody, or antigen binding fragment thereof, of claims 1-8 wherein said antibody binds to a cytokine

12. The antibody, or antigen binding fragment thereof, of claim 10 wherein said virus is a Rous Sarcoma Virus, an Ebola Virus, a Human Immunodeficiency Virus, or an Influenza Virus.

13. The antibody, or antigen binding fragment thereof, of claims 1-8 wherein said antibody is produced in a mammalian cell.

14. The antibody, or antigen binding fragment thereof, of claims 1-8 wherein said antibody is produced in a yeast cell.

15. The antibody, or antigen binding fragment thereof, of claim 13 wherein said antibody is produced in a CHO cell.

16. The antibody or antigen binding fragment of claims 1-8 wherein said antibody is produced in a plant cell.

17. A composition comprising an antibody or antigen binding fragment of claims 1-8 and plant material.

18. The composition of claim 17, wherein said plant material is selected from the group consisting of plant cell wall, plant organelle, plant cytoplasm, plant protoplast, plant cell, intact plant, viable plant, plant leaf extract, plant leaf homogenate, and chlorophyll.

19. The antibody, or antigen binding fragment thereof, of claims 1-8, wherein the antibody dissociates from any of the proteins selected from the group consisting of FcγRI and FcγRIII and wherein Kd from FcγRI is 1×10⁻⁸ M or less and the Kd from FcγRIII is 1×10⁻⁷ M or less.

20. The antibody, or antigen binding fragment thereof, of claims 1-8, wherein the heavy chain constant region is selected from the group consisting of IgA, IgD, IgE, IgG, and IgM.

21. The monoclonal antibody of claims 1-8, wherein the heavy chain constant region is an IgG₁.

22. An isolated nucleic acid encoding the GNGN or G1/G2 antibody, or antigen binding fragment thereof, of claims 1-8.

23. An isolated cell, comprising the antibody or antigen-binding fragment of any of claims 1-8 or the nucleic acid of claim 22.

24. A method of expression of an isolated antibody or antigen-binding fragment thereof, comprising culturing the cell of claim 23 under conditions which express and glycosylate the encoded antibody.

25. The cell of claim 24, wherein the cell is a plant cell.

26. The cell of claim 25, wherein the plant cell is from N. benthamiana.

27. The cell of claim 26, wherein the plant cell has been modified by RNAi or gene knockout to eliminate expression of plant-specific xylosyl and fucosyl transferase genes.

28. A pharmaceutical composition comprising the GNGN or G1/G2 antibody, or antigen binding fragment thereof, of claims 1-8 and a pharmaceutically acceptable carrier.

29. A method of treating a subject infected with a virus comprising administering to the subject a therapeutically effective amount of the composition of claim 38, wherein the antibody, or antigen binding fragment thereof, recognizes and binds to the virus.

30. A method of treating a subject with cancer comprising administering to the subject a therapeutically effective amount of the composition of claim 38, wherein the antibody, or antigen binding fragment thereof, recognizes and binds to cancer cells.

31. A method of treating a subject with an inflammatory disease comprising administering to the subject a therapeutically effective amount of the composition of claim 38, wherein the antibody, or antigen binding fragment thereof, recognizes and binds to the inflammatory antigen.

32. A kit comprising the antibody or antigen-binding fragment of any of claims 1-8, in one or more containers, and instructions for use.
